(19) United States
(12) Patent Application Publication
(10) Pub. No.: US 2021/0285933 A1
(43) Pub. Date: Sep. 16, 2021

(54) HIGH THROUGHPUT DRUG SCREENING OF CANCER STEM CELLS

(71) Applicant: UNIVERSITY OF WASHINGTON, Seattle, WA (US)

(72) Inventors: Pamela BECKER, Seattle, WA (US); Su-In LEE, Seattle, WA (US); Carl BLAU, Seattle, WA (US); Vivian OEHLER, Seattle, WA (US); Timothy MARTINS, Seattle, WA (US); Raymond J. MONNAT, Jr., Seattle, WA (US)

(73) Assignee: UNIVERSITY OF WASHINGTON, Seattle, WA (US)

(21) Appl. No.: 17/257,945

(22) PCT Filed: Jul. 3, 2019
(86) PCT No.: PCT/US19/40617

§ 371 Date: Jan. 5, 2021

Related U.S. Application Data
(60) Provisional application No. 62/694,874, filed on Jul. 6, 2018.

(51) Int. Cl. G01N 33/50

(52) U.S. Cl. CPC G01N 33/5011; G01N 2500/10; G01N 33/5073

(57) ABSTRACT

Described herein are functional cell assays and methods for selecting a personalized anti-cancer treatment regimen that can improve treatment of cancer in a subject, identify resistance of the subject's cancer to one or more anti-cancer agents and/or validate the current drug treatment strategy.

### CROSS REFERENCE TO RELATED APPLICATIONS

This application claims benefit under 35 U.S.C. § 119(e) of U.S. Provisional Application No. 62/694,874, filed Jul. 6, 2018, the contents of which are incorporated herein by reference in its entirety.

### STATEMENT OF FEDERALLY SPONSORED RESEARCH AND DEVELOPMENT

This invention was made with government support under Grant No. P01 CA077852 awarded by the National Institutes of Health (NIH). The government has certain rights in the invention.

### FIELD OF THE INVENTION

The methods and assays described herein relate to the personalized treatment of cancer.

### BACKGROUND

Cancer stem cells are a small, distinct subset of cells within each tumor (or blood cancer) capable of indefinite self-renewal. Resistance to anti-cancer agents has been proposed to be due, in part, to resistance of such cancer stem cells (CSC). Thus, even despite successful treatment of an acute cancer with chemotherapy and/or radiation, CSCs can persist and grow into secondary tumors, metastases or be responsible for relapse of the original cancer.

To improve cancer treatments and prevent relapse/resistance, it is helpful to include an agent to which the cancer stem cell is not resistant.

### SUMMARY

Cancer heterogeneity refers to the characteristics of certain cancers, where the tumor or cancer comprises heterogeneous cells with a variety of different phenotypes, including variations in the responsiveness of such cells to anti-cancer therapies. Given that cancers can vary so widely on the cellular level, it follows that the same cancer in two different patients can have a very different responsiveness/resistance profile to a given anti-cancer agent or chemotherapeutic. Thus, in order to determine the effectiveness of a given therapeutic in a given patient with a given cancer, it is beneficial to test, functionally, the effect of such a therapeutic in a personalized manner, thereby permitting precision therapeutic treatment of the cancer.

Described herein are functional cell assays and methods for selecting a personalized anti-cancer agent regimen that can improve treatment of cancer in a subject, identify resistance of the subject's cancer to one or more anti-cancer agents and/or validate the current anti-cancer treatment strategy.

Provided herein in one aspect is a high throughput functional cell assay comprising the steps of: (a) contacting aliquots of a biological sample from an individual having cancer each with individual members of a panel of therapeutic drugs or a combination thereof, the sample comprising a population of non-stem cell cancer cells and a population of cancer stem cells (CSCs); and (b) quantifying, respectively, cell viability of the population of CSCs and the population of non-stem cell cancer cells (NSCCCs).

Another aspect described herein relates to a high throughput functional cell assay comprising the steps of: (a) contacting aliquots of a biological sample from an individual having cancer with individual members of a panel of anti-cancer agents, the sample comprising a population of non-stem cell cancer cells and a population of cancer stem cells (CSCs); and (b) quantifying, respectively, cell viability of the population of CSCs and the population of non-stem cell cancer cells (NSCCCs).

In one embodiment of this aspect and all other aspects described herein, cell viability is assessed using a tetrazolium reduction assay, a resazurin reduction assay, a protease viability marker assay, a live cell protease assay, an ATP assay, a luciferase-based real-time assay, flow cytometry, or high content imaging.

In another embodiment of this aspect and all other aspects provided herein, the assay further comprises a step, performed prior to steps (a) and (b), of seeding the aliquots of the biological sample in a plurality of wells.

In another embodiment of this aspect and all other aspects provided herein, the panel of anti-cancer agents comprises at least two drugs selected from a class or classes of drugs selected from the group consisting of: tyrosine kinase inhibitors, histone deacetylase inhibitors, cyclin-dependent kinase inhibitors, proteasome inhibitors, targeted inhibitor, PARP inhibitors, alkylating agents, cisplatinum compounds, anthracyclines, topoisomerase inhibitors, Flt-3 kinase inhibitors, and microtubule assembly inhibitors.

In another embodiment of this aspect and all other aspects provided herein, the panel of anti-cancer agents comprises at least 2 anti-cancer agents and no more than 1000 anti-cancer agents.

In another embodiment of this aspect and all other aspects provided herein, each individual member of the panel is tested with at least five different concentrations of the anti-cancer agent for each population.

In another embodiment of this aspect and all other aspects provided herein, a dose-response curve is generated for each individual member and each population using the data from the at least two different concentrations of the anti-cancer agent. One of skill in the art will recognize that the reliability of a dose-response curve for EC50 or IC50 measurements increases as the number of different concentrations of an anti-cancer drug tested also increases. Thus in some preferred embodiments, a dose-response curve is generated for each individual member and each population using the data from at least 3, at least, 4, at least 5, at least 6, at least 7, at least 8, at least 9, at least 10, at least 12, at least 15, at least 20 different concentrations or more.

In another embodiment of this aspect and all other aspects provided herein, an EC50 or IC50 is determined for a given anti-cancer agent is determined from the dose-response curve for that anti-cancer agent in each cell population.

In another embodiment of this aspect and all other aspects provided herein, an EC50 for a given anti-cancer agent that is at least ten-fold lower than the maximal plasma concentration in humans indicates that the cells are susceptible to that anti-cancer agent and/or the anti-cancer agent is considered for use in the treatment of the subject's cancer.

In another embodiment of this aspect and all other aspects provided herein, Area Under the Curve (AUC) is calculated for each individual member in the panel of anti-cancer agents and for each population from the respective dose-response curve for that anti-cancer agent.

In another embodiment of this aspect and all other aspects provided herein, the biological sample is obtained from the subject using a resection, biopsy, vacuum assisted biopsy, core needle biopsy or fine needle aspirate of a primary or metastatic tumor, or wherein the biological sample comprises a blood sample, a bone marrow aspiration, cerebrospinal fluid, pleural fluid, pericardial fluid, ascites fluid, urine, or isolated cells thereof.

In another embodiment of this aspect and all other aspects provided herein, the assay further comprises a step of ranking the individual members of the panel of anti-cancer agents based on their effect on cell viability for CSCs and/or NSCCCs.

In another embodiment of this aspect and all other aspects provided herein, the assay further comprises a step of comparing the cell viability for each anti-cancer agent on CSCs and/or NSCCCs to a reference.

In another embodiment of this aspect and all other aspects provided herein, quantifying step (b) comprises detecting signal from one or more markers permitting quantitative distinction between populations of CSCs and NSCCCs.

In another embodiment of this aspect and all other aspects provided herein, the assay further comprises a step of contacting the population of NSCCCs and the population of cancer stem cells (CSCs) with detectable probes that specifically bind and provide signal for the one or more markers.

In another embodiment of this aspect and all other aspects provided herein, the signal comprises fluorescent emission.

In another embodiment of this aspect and all other aspects provided herein, the markers permitting quantitative distinction between populations of CSCs and NSCCCs are selected from the markers in Table 1 or 2.

Also provided herein, in another aspect, is a method for selecting a personalized treatment for a subject having cancer, the method comprising: (a) performing a high throughput functional cell assay of any one of claims 1-15 on a biological sample from a subject having cancer; and (b) on the basis of cell viability determined for CSCs and NSCCCs in (a), selecting a combination of at least two anti-cancer agents from the panel, the combination comprising a drug(s) effective to kill CSCs and a drug(s) effective to kill NSCCCs, thereby selecting a personalized treatment for the subject.

In one embodiment of this aspect and all other aspects provided herein, the assay further comprises administering the combination of anti-cancer agents to the subject, thereby treating the subject's cancer.

In another embodiment of this aspect and all other aspects provided herein, the cancer is refractory to or the subject has relapsed from prior treatment with a given anti-cancer agent.

In another embodiment of this aspect and all other aspects provided herein, the CSCs comprise a leukemic stem cell, an acute myeloid leukemia stem cell, a brain cancer stem cell, a breast cancer stem cell, an ovarian cancer stem cell, a pancreatic cancer stem cell, a prostate cancer stem cell, a melanoma stem cell, a multiple myeloma stem cell, a colon cancer stem cell, an esophageal cancer stem cell, a stomach cancer stem cell, a lung cancer stem cell, a liver cancer stem cell, a head and neck squamous cell carcinoma stem cell, multiple myeloma stem cell, or a non-melanoma skin cancer stem cell.

Another aspect provided herein relates to a high throughput functional cell assay comprising the steps of: (a) isolating from a biological sample obtained from a subject having cancer, a population enriched for viable cancer stem cells (CSCs), (b) isolating from the same or a different biological sample obtained from the subject having cancer, a population enriched for viable non-stem cell cancer cells (NSCCCs); (c) contacting aliquots of the population enriched for CSCs with individual members of a panel of anti-cancer agents, (d) contacting aliquots of the population enriched for NSCCCs with individual members of a panel of anti-cancer agents, and (e) determining cell viability of the cells in each of the populations of step (c) and (d).

In one embodiment of this aspect and all other aspects provided herein, cell viability is assessed using a tetrazolium reduction assay, a resazurin reduction assay, a protease viability marker assay, a live cell protease assay, an ATP assay, a luciferase-based real-time assay, flow cytometry, or high content imaging.

In another embodiment of this aspect and all other aspects provided herein, the assay further comprises a step, performed prior to steps (c) and (d), of seeding CSCs in a first plurality of wells and a step of seeding NSCCCs in a second plurality of wells.

In another embodiment of this aspect and all other aspects provided herein, the panel of anti-cancer agents comprises at least two drugs selected from a class or classes of drugs selected from the group consisting of: tyrosine kinase inhibitors, histone deacetylase inhibitors, cyclin-dependent kinase inhibitors, proteasome inhibitors, targeted inhibitor, PARP inhibitors, alkylating agents, cisplatinum compounds, anthracyclines, topoisomerase inhibitors, Flt-3 kinase inhibitors, and microtubule assembly inhibitors.

In another embodiment of this aspect and all other aspects provided herein, the panel of anti-cancer agents comprises at least 2 anti-cancer agents and no more than 1000 anti-cancer agents.

In another embodiment of this aspect and all other aspects provided herein, each individual member of the panel is tested in at least two different concentrations of the anti-cancer agent for each population.

In another embodiment of this aspect and all other aspects provided herein, a dose-response curve is generated for each individual member and each population using the data from the at least five different concentrations of the anti-cancer agent.

In another embodiment of this aspect and all other aspects provided herein, Area Under the Curve (AUC) is calculated for each individual member in the panel of anti-cancer agents and for each population from the respective dose-response curve for that anti-cancer agent.

In another embodiment of this aspect and all other aspects provided herein, the biological sample is obtained from the subject using a resection, biopsy, vacuum assisted biopsy, core needle biopsy or fine needle aspirate of a primary or metastatic tumor or wherein the biological sample comprises a blood sample, a bone marrow aspiration, cerebrospinal fluid, pleural fluid, pericardial fluid, ascites fluid, urine, or isolated cells thereof.

In another embodiment of this aspect and all other aspects provided herein, the assay further comprises a step of ranking the individual members of the panel of anti-cancer agents based on their effect on cell viability for each population.

In another embodiment of this aspect and all other aspects provided herein, the assay further comprises a step of comparing the cell viability for each anti-cancer agent and for each population to a reference.

Also provided herein, in another aspect, is a method for selecting a personalized treatment for a subject having cancer, the method comprising: (a) isolating from a biological sample obtained from a subject having cancer, a population enriched for cancer stem cells (CSCs), (b) isolating from the same or a different biological sample obtained from the subject having cancer, a population enriched for non-stem cell cancer cells (NSCCCs); (c) contacting aliquots of the population enriched for CSCs with individual members of a panel of anti-cancer agents, (d) contacting aliquots of the population enriched for cancer cells with individual members of a panel of anti-cancer agents or combinations thereof, (e) determining cell viability of the cells in each of the populations of step (c) and (d), (f) selecting, based on criteria comprising reduced cell viability, at least one anti-cancer agent, thereby selecting a personalized treatment for the subject having cancer.

In one embodiment of this aspect and all other aspects provided herein, the method further comprises administering the at least one anti-cancer agent selected in step (f) to the subject having cancer.

In another embodiment of this aspect and all other aspects provided herein, the method further comprises a step, performed prior to steps (c) and (d), of seeding CSCs in a plurality of wells and a step of seeding cancer cells in a plurality of wells.

In another embodiment of this aspect and all other aspects provided herein, the panel of anti-cancer agents comprises at least two drugs selected from a class or classes of drugs selected from the group consisting of: tyrosine kinase inhibitors, histone deacetylase inhibitors, cyclin-dependent kinase inhibitors, proteasome inhibitors, targeted inhibitor, PARP inhibitors, alkylating agents, cisplatinum compounds, anthracyclines, topoisomerase inhibitors, Flt-3 kinase inhibitors, and microtubule assembly inhibitors.

In another embodiment of this aspect and all other aspects provided herein, wherein the panel of anti-cancer agents comprises at least 2 anti-cancer agents and no more than 1000 anti-cancer agents.

In another embodiment of this aspect and all other aspects provided herein, the method further comprises a step of obtaining the biological sample from the subject having cancer.

In another embodiment of this aspect and all other aspects provided herein, the cancer is refractory to or the subject has relapsed from prior treatment with a given anti-cancer agent.

In another embodiment of this aspect and all other aspects provided herein, the CSCs comprise a leukemic stem cell, an acute myeloid leukemia stem cell, a brain cancer stem cell, a breast cancer stem cell, an ovarian cancer stem cell, a pancreatic cancer stem cell, a prostate cancer stem cell, a melanoma stem cell, a multiple myeloma stem cell, a colon cancer stem cell, an esophageal cancer stem cell, a stomach cancer stem cell, a lung cancer stem cell, a liver cancer stem cell, a head and neck squamous cell carcinoma stem cell, multiple myeloma stem cell, or a non-melanoma skin cancer stem cell.

In another embodiment of this aspect and all other aspects provided herein, the biological sample lacks red blood cells.

In another embodiment of this aspect and all other aspects provided herein, the method further comprises a step of removing red blood cells.

In another embodiment of this aspect and all other aspects provided herein, the method further comprises a step of ranking the individual members of the panel of anti-cancer agents based on their effect on cell viability for each population.

In another embodiment of this aspect and all other aspects provided herein, the method further comprises a step of comparing the cell viability for each anti-cancer agent and for each population to an appropriate reference.

In another embodiment of this aspect and all other aspects provided herein, each individual member of the panel is tested with at least two different concentrations of the anti-cancer agent in each population.

In another embodiment of this aspect and all other aspects provided herein, a dose-response curve is generated for each individual member and each population using the data from the at least five different concentrations of the anti-cancer agent.

In another embodiment of this aspect and all other aspects provided herein, Area Under the Curve (AUC) is calculated for each individual member in the panel of anti-cancer agents and for each population from the respective dose-response curve for that anti-cancer agent.

In another embodiment of this aspect and all other aspects provided herein, the biological sample is obtained from the subject using a resection, biopsy, vacuum assisted biopsy, core needle biopsy or fine needle aspirate of a primary or metastatic tumor or wherein the biological sample comprises a blood sample, a bone marrow aspiration, cerebrospinal fluid, pleural fluid, pericardial fluid, ascites fluid, urine, or isolated cells thereof.

In another embodiment of this aspect and all other aspects provided herein, step (f) is performed by a skilled clinician.

In another embodiment of this aspect and all other aspects provided herein, the results of step (f) are communicated to a skilled clinician.

In another embodiment of this aspect and all other aspects provided herein, the population enriched for non-stem cell cancer cells comprises less than 20% CSCs and/or wherein the population enriched for CSCs comprises less than 20% NSCCs.

In another embodiment of this aspect and all other aspects provided herein, the steps (a)-(f) are repeated at least once.

In another embodiment of this aspect and all other aspects provided herein, the steps (a)-(g) are repeated in the presence of a different panel of anti-cancer agents.

Another aspect provided herein relates to a method for monitoring treatment efficacy in a subject being treated for cancer, the method comprising: (a) isolating from a biological sample obtained from a subject being treated for cancer with an anti-cancer agent, a population enriched for cancer stem cells (CSCs), (b) isolating from the same or a different biological sample obtained from the subject being treated for cancer with an anti-cancer agent(s), a population enriched for cancer cells; (c) contacting aliquots of the population enriched for CSCs with the anti-cancer agent(s), (d) contacting aliquots of the population enriched for non-stem cell cancer cells with the anti-cancer agent(s), and (e) determining cell viability of the cells in each of the populations of step (c) and (d), wherein a reduction in cell viability in the presence of the anti-cancer agent as compared to an untreated or vehicle treated aliquot of the same cell population indicates that the anti-cancer agent is efficacious in the subject being treated for cancer.

In one embodiment of this aspect and all other aspects provided herein, the method is repeated at least once while the subject is being treated for cancer.

In another embodiment of this aspect and all other aspects provided herein, the method is repeated weekly, monthly, every 6 months, or annually.

The method of any one of claims 53-55, further comprising the steps of: (a) contacting aliquots of the population enriched for CSCs with individual members of a panel of anti-cancer agents, (b) contacting aliquots of the population enriched for cancer cells with individual members of a panel of anti-cancer agents, (c) determining cell viability of the cells in each of the populations of step (a) and (b), (d) selecting, based on criteria comprising reduced cell viability assessed in step (c), the same or a different anti-cancer agent or combination thereof than the anti-cancer agent used to treat cancer in the subject.

Another aspect provided herein relates to a method for selecting a treatment for or treating a subject having acute myeloid leukemia (AML), the method comprising: (a) isolating from a biological sample comprising white blood cells obtained from a subject having AML, a population enriched for leukemic stem cells (LSCs), (b) isolating from the same or a different biological sample comprising white blood cells obtained from the subject having AML, a population enriched for blast cells; (c) contacting aliquots of the population enriched for LSCs with individual members of a panel of anti-cancer agents or a combination thereof to determine the susceptibility of the LSCs to each drug or combination of drugs, (d) contacting aliquots of the population enriched for blast cells with individual members of a panel of anti-cancer agents or a combination thereof to determine the susceptibility of the blast cells to each drug or combination thereof, (e) selecting at least one drug from step (c) and/or step (d) to which the LSCs and/or blast cells are determined to be susceptible, and (f) optionally administering the at least one drug selected in step (e) to the subject having AML, thereby treating the subject having AML.

In one embodiment of this aspect and all other aspects provided herein, the method further comprises a step, performed prior to steps (c) and (d), of seeding LSCs in a plurality of wells and a step of seeding blast cells in a plurality of wells.

In another embodiment of this aspect and all other aspects provided herein, the panel of anti-cancer agents comprises at least two drugs selected from a class or classes of drugs selected from the group consisting of: tyrosine kinase inhibitors, histone deacetylase inhibitors, cyclin-dependent kinase inhibitors, proteasome inhibitors, targeted inhibitor, PARP inhibitors, alkylating agents, cisplatinum compounds, anthracyclines, topoisomerase inhibitors, Flt-3 kinase inhibitors, and microtubule assembly inhibitors.

In another embodiment of this aspect and all other aspects provided herein, the panel of anti-cancer agents comprises at least 2 anti-cancer agents and no more than 1000 anti-cancer agents.

In another embodiment of this aspect and all other aspects provided herein, the method further comprises a step of obtaining the biological sample comprising white blood cells from a subject having AML.

In another embodiment of this aspect and all other aspects provided herein, the subject having AML is refractory to or has relapsed from conventional AML treatment.

In another embodiment of this aspect and all other aspects provided herein, the conventional AML treatment comprises treatment with cytarabine and an anthracycline.

In another embodiment of this aspect and all other aspects provided herein, the biological sample comprising white blood cells is a CD34+ enriched blast cell population.

In another embodiment of this aspect and all other aspects provided herein, at least 75% of the cells in the CD34+ enriched blast cell population are CD34+ blast cells.

In another embodiment of this aspect and all other aspects provided herein, the biological sample lacks red blood cells.

In another embodiment of this aspect and all other aspects provided herein, the method further comprises a step of removing red blood cells.

In another embodiment of this aspect and all other aspects provided herein, each anti-cancer agent in the panel of anti-cancer agents is tested for each population using at least two different concentrations of the anti-cancer agent.

In another embodiment of this aspect and all other aspects provided herein, each anti-cancer agent in the panel of anti-cancer agents is tested for each population using at least five different concentrations of the anti-cancer agent.

In another embodiment of this aspect and all other aspects provided herein, a dose-response curve is generated for each population using the data from the at least five different concentrations of the anti-cancer agent.

In another embodiment of this aspect and all other aspects provided herein, Area Under the Curve (AUC) is calculated for each anti-cancer agent in the panel of drugs for each population from the respective dose-response curve for that anti-cancer agent.

In another embodiment of this aspect and all other aspects provided herein, the method further comprises comparing the AUC for each anti-cancer agent in the panel of drugs for each population to an AUC calculated from the dose-response curve of mitomycin C for the same population.

In another embodiment of this aspect and all other aspects provided herein,
(i) an AUC^{stem mean}>AUC^{blast mean} for an anti-cancer agent in the panel indicates that the blast cells are susceptible to the anti-cancer agent, or
(i) an AUC^{stem mean}<AUC^{blast mean} for an anti-cancer agent in the panel indicates that the LSC cells are susceptible to the anti-cancer agent.

In another embodiment of this aspect and all other aspects provided herein, step (e) is performed by a skilled clinician.

In another embodiment of this aspect and all other aspects provided herein, the results of step (e) are communicated to a skilled clinician.

In another embodiment of this aspect and all other aspects provided herein, the population enriched for LSCs comprises at least 75% LSC cells comprising a marker profile of CD34⁺ CD38^{lo/-} CD123⁺.

In another embodiment of this aspect and all other aspects provided herein, the population enriched for blast cells comprises less than 20% LSCs and/or the population enriched for LSCs comprises less than 20% blast cells.

In another embodiment of this aspect and all other aspects provided herein, the biological sample comprising white blood cells is fractionated by FACS.

In another embodiment of this aspect and all other aspects provided herein, the method further comprises a step of fractionating the biological sample comprising white blood cells for viable cells by the characteristics of CD45^{dim}, side scatter^{lo}.

In another embodiment of this aspect and all other aspects provided herein, the method is repeated at least once.

In another embodiment of this aspect and all other aspects provided herein, the method is repeated at least once using a different panel of anti-cancer agents.

### BRIEF DESCRIPTION OF FIGURES

FIGS. 1A-1C show data relating to the susceptibility of leukemic stem cells (LSCs) and Acute Myeloid Leukemia (AML) blast cells to chemotherapeutic agents. FIG. 1A shows data relating to the drug sensitivity of AML blasts versus leukemia stem cells (LSCs). A volcano plot highlighting compounds with significantly greater drug response for blasts (AUCstem mean>AUCblast mean, P-Value<0.1) or LSCs (AUCstem mean<AUCblast mean, P-Value<0.1). Each circle or diamond represents a different compound. The findings are based on aggregate data using 4 patient samples. Notice that the drugs commonly used in AML (diamonds) tend to be more effective for blasts than LSCs. FIG. 1B is a paired heat map indicating that blast cell specific compounds are more effective against blasts than LSCs. Heat map graphs use AUCstem mean and AUCblast mean data to capture relative response to the blast specific drugs. FIG. 1C shows exemplary dose response curves for two of our patient samples. On the left, note that the patients LSCs were sensitive to YM-155 and gemcitabine but resistant to cytarabine and idarubicin; on the right LSCs were sensitive to AMG-900 and Sunitinib but resistant to cytarabine and daunorubicin.

FIGS. 2A-2B Xenograft model demonstrates drug resistance in clonal evolution. FIG. 2A show exemplary dose response curves of the pre-engraftment AML blasts and the engrafting subclone. FIG. 2B is a heat map comparing relative degree of inhibition of the pre-engraftment AML blast population and the engrafting subclone. Dark grey/black: cell death, medium grey: cell survival. Notice that the engrafting subclone was resistant to chemotherapy agents used to treat AML today.

FIG. 3 LSCs resistance to mitomycin C may indicate differential response to DNA damage. Dose response curves describing mitomycin C's effect on blasts vs. LSCs. Notice that for both patient samples, the blasts are sensitive as expected but the LSCs are resistant. Error bars represent the standard deviation from 8 or 4 determinations for blasts or LSCs, respectively.

FIG. 4 shows data indicating that drug susceptibility patterns are distinct in each patient. An exemplary heat map depicts in vitro high throughput screening drug response for individual patient samples separated by effect vs. blasts and effect vs. LSCs. Drug response is based on AUCstem and AUCblast data, with 10 drugs commonly used in AML mapped here as examples. Medium grey: cell death, Dark grey/black: cell survival. Note that each patient's stem cells exhibit a unique pattern of drug susceptibility compared to other patients in the cohort; the same is true for the blast cells. Further, each individual patient's LSC drug susceptibility pattern is different than his/her blast cell susceptibility pattern, further emphasizing the need for HTS to assess the response patterns for both cell fractions.

FIG. 5 shows data relating to FACS analysis of engraftment of NODscid IL2R gc−/− mice at week 3. The x-axis represents hCD45 and the y-axis represents mCD45. The circled graph represents the “rapid engrafter.” Note the robust degree of engraftment when compared to the other xenografts.

FIG. 6 is a table showing the clinical characteristics for 5 AML patient samples.

FIGS. 7A-7B are schematics relating to exemplary experiments using patient samples (AML-190, AML-211, AML-228, AML-237; FIG. 7A), and NOD/SCID IL2R γc^{-/-} mouse engraftment sample (AML-153, FIG. 7B).

FIGS. 8A-8B show exemplary heatmaps of Area under the curve (AUC) data for leukemia blasts (FIG. 8A) and stem cells (FIG. 8B) as compared to normal CD34+ cells. Notice that normal CD34+ cells are most sensitive (Dark grey/black) to anti-cancer compounds when compared to blasts and LSCs (Medium grey).

FIG. 9 is a table depicting plasma concentrations with typical dosing of drugs used in acute myeloid leukemia. KEY: regular text: blast IC50<predicted plasma concentration and stem IC50>predicted plasma concentration; italicized font: blast and stem IC50 less than predicted plasma concentration; bold font:blast and stem IC50>predicted plasma concentration; *cytarabine based on comparison to peak plasma concentration

### DETAILED DESCRIPTION

The methods described herein are based, in part, upon the recognition that cancer cell heterogeneity and the ensuing difficulty in cancer treatment can be addressed by methods that identify, on a patient-specific and cancer-specific basis, those agents that are effective to kill not just bulk tumor cells, but also cancer stem cells for a given patient's cancer. The differential detection and measurement of NSCCCs and CSCs in a sample, coupled with any of a number of different cell viability assays, permits the application of high-throughput approaches to the identification of the agent or agents that most effectively target a given subject's cancer. The following describes considerations necessary to perform the methods permitting such identification and therapies based upon it.

### Definitions

As used herein, the term “cancer stem cell(s)” refers to a cell(s) that can be a progenitor of a highly proliferative cancer cell. A cancer stem cell (CSC) has the ability to re-grow a tumor as demonstrated by its ability to form tumors in immunocompromised mice, and typically to form tumors upon subsequent serial transplantation in immunocompromised mice. Cancer stem cells are also typically slow-growing relative to the bulk of a tumor; that is, cancer stem cells are generally quiescent. In certain embodiments, but not all, the cancer stem cell may represent approximately 0.1 to 10% of a tumor. It is specifically contemplated herein that cancer stem cells can have a different sensitivity to a given anti-cancer agent than non-stem cell cancer cells (NSCCCs). In other embodiments, a CSC comprises expression of at least one stem cell marker or does not comprise expression of at least one marker present in differentiated cells or in NSCCCs from the same tumor or same tumor type.

As used herein, the term “NSCCCs” refers to cancer cells obtained from a subject that substantially lack cancer stem cell activity; thus they are not capable of re-growing a tumor. In some embodiments, an NSCCC comprises expression of at least one marker associated with the endogenous cells from which they are derived and/or lacks expression of at least one stem cell marker.

The term “marker” as used herein is used to describe a characteristic and/or phenotype of a cell. Markers can be used for selection of cells comprising characteristics of interest and can vary with specific cells. Markers are characteristics, whether morphological, structural, functional or biochemical (enzymatic) characteristics of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of certain proteins, lipids etc. Where the absence of a marker is characteristic of a given cell or cancer cell type, the cell or cancer is generally also positive for other markers, Thus, markers can be a combination of a set of unique characteristics or the presence and/or absence of polypeptides and other morphological or structural characteristics. In one embodiment, the marker is a cell surface marker (e.g., a stem cell marker or a non-stem cell marker). In one embodiment, the cell surface phenotype may be determined by detecting the expression of a combination of cell surface antigens. Non-limiting examples of cell surface phenotypes of cancer stem cells of certain tumor types include CD34+/CD38−, CD123+, CD44+/CD24−, CD133+, CD34+/CD10−/CD19−, CD138−/CD34−/CD19+, CD133+/RC2+, CD44+/α2β1hi/CD133+, CLL-1, SLAMs, and other cancer stem cell surface phenotypes mentioned herein, as well as those that are known in the art. In certain embodiments, a phenotype can be assessed by the loss (or lack) of expression of a given marker.

The term “substantially pure,” or “homogeneous” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “homogeneous,” with regard to a population of cancer stem cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not cancer stem cells (i.e., non-stem cell cancer cells).

The terms “enriching” or “enriched” are used interchangeably herein and mean that the yield (fraction) of cells of one type, such as cancer stem cells for use in the methods described herein, is increased by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in a starting biological sample, culture, or preparation.

The term “separation” or “selection” as used herein refers to isolating different cell types into one or more populations and collecting the isolated population(s) as a target cell population(s) which is enriched, for example, in a specific target cell (e.g., cancer stem cell or non-stem cell cancer cell). Selection can be performed using positive selection, whereby a target enriched cell population is retained in one fraction, or negative selection, whereby non-target cell types are removed from the fraction (thereby enriching for desired target cell types in the remaining cell population).

The term “positive selection” as used herein refers to selection of a desired cell type by retaining the cells of interest. In some embodiments, positive selection involves the use of an agent to assist in retaining the cells of interest, e.g., use of a positive selection agent such as an antibody which has specific binding affinity for a surface antigen on the desired or target cell. In some embodiments, positive selection can occur in the absence of a positive selection agent, e.g., in a “touch-free” or closed system, for example, where positive selection of a target cell type is based on any of cell size, density and/or morphology of the target cell type. It is specifically contemplated herein that the remaining cells that are not selected for are retained as a separate fraction or sample. For example, a biological sample can be selected for cancer stem cells, while the remaining, non-selected cells are retained as an enriched population of non-stem cell cancer cells, and vice versa.

The term “negative selection” as used herein refers to selection of non-target cells (e.g., NSCCCs, or CSCs) and removal of such non-target cells from the “enriched population” of a given cell type, thereby retaining (and thus enriching) the desired target cell type in a fraction or sample. It is specifically contemplated herein that the remaining cells that are not selected for are retained as an enriched fraction of the non-selected cells in a separate sample. For example, a biological sample can be selected for cancer stem cells, while the remaining, non-selected cells are retained as a population enriched for non-stem cell cancer cells, and vice versa. In some embodiments, negative selection involves the use of an agent to assist in selecting undesirable cells for removing, e.g., by use of a negative selection agent such as a monoclonal antibody which has specific binding affinity for a surface antigen on unwanted or non-target cells. In some embodiments, negative selection does not involve a negative selection agent. In some embodiments, negative selection can occur in the absence of a negative selection agent, e.g., in a “touch-free” or closed system, for example, where negative selection of an undesired (non-target) cell type to be removed from an enriched population of the target cell is based on any of cell size, density and/or morphology of the undesired (non-target) cell type.

As used herein, the term “panel of anti-cancer agents” refers to a compiled set of desired anti-cancer agents that can be measured in parallel for activity in a functional cell assay as described herein. At a minimum, a panel of anti-cancer agents comprises at least two different anti-cancer agents. A panel of anti-cancer agents can comprise any desired combination of anti-cancer agents and can include, for example, multiple classes of drugs and/or multiple drugs within a class. In some embodiments, the panel of anti-cancer agents is specific to a given cancer, i.e., designed to include drugs that are known to have some degree of effect against a given cancer. Such panels can be referred to as e.g., a “breast cancer-specific panel of anti-cancer agents,” a “prostate cancer-specific panel of anti-cancer agents,” or an “acute myeloid leukemia-specific panel of anti-cancer agents,” and the like. It should be understood that a panel “specific” for a given cancer type can include drugs that also have efficacy against one more additional types of cancer, even if they are particularly effective against the specified type. In theory, a panel of anti-cancer agents can include every drug known, approved, or in consideration for approval at any given time. However, panel size can be dependent on the format used to detect the effects of the drugs. As but one example, the size of a microtiter plate, and the number of concentrations at which each drug is tested will determine the maximal number of different agents to be included in a given panel. Of course, a panel could be comprised of those drugs arranged in more than one microtiter plate; as a non-limiting example, where format for read-out calls for 96 well microtiter plates, the panel could be comprised in two such plates, for a total of up to 192 different drugs at a single dose or concentration per drug. In practice, it can be beneficial to include a plurality of different concentrations of each drug of a panel, which can reduce the number of drugs that can be screened simultaneously but which provides additional information regarding sensitivity of the tested cancer's cells to the drugs in the panel. In one embodiment, the maximal number of individual members of a panel of anti-cancer agents is 1000 (e.g., 900 or less, 800 or less, 700 or less, 600 or less, 50 or less, 400 or less, 300 or less, 200 or less, 100 or less, 50 or less, 25 or less, 20 or less, 5 or less, 4 or less, or 3 or less). It should be understood that one drug e.g., in a plurality of different concentrations, does not constitute a panel as described herein. A panel can include, for example, at least 3 different drugs, at least 4 different drugs, at least 5 different drugs, at least 6, 7, 8, 9, 10, 15, 20, 25, 50, 75, 100, 125, 150, 175, 200, 225, 250, 275, 300, 325, 350, 375, 400, 425, 450 or at least 475 drugs or more.

As used herein, the term “anti-cancer agent” applies to a drug or agent effective for the treatment of cancer. It should be understood that an anti-cancer agent includes not only small molecule cytotoxic agents but also, for example, tyrosine kinase inhibitors, histone deacetylase inhibitors, cyclin-dependent kinase inhibitors, proteasome inhibitors, targeted inhibitors, PARP inhibitors, topoisomerase inhibitors, Flt-3 kinase inhibitors, and microtubule assembly inhibitors.

As used herein, the term “targeted inhibitor” refers to a compound or agent that inhibits the action of a given class of pathways, biological molecules and/or enzymes (e.g., MEK inhibitors) that are involved in cancer.

As used herein, the term “individual member” refers to a single anti-cancer agent in a panel of agents as described herein. Thus, an “individual member” refers to a distinct agent or drug that differs from the other agents or drugs in the panel. The term “individual member” does not encompass different concentrations of a single anti-cancer agent. As used herein, the term “or combination thereof” when used in reference to members of a panel of anti-cancer agents refers to a combination of agents that are administered simultaneously to a given aliquot to determine the combined action of the agents on susceptibility of the cells.

As used herein, the terms “separate aliquots,” “aliquots” and “plurality of aliquots” are used interchangeably herein and refer to a plurality of biological samples obtained from a single subject. In one embodiment, the separate aliquots are sub-samples of a single biological sample from a single subject. It is contemplated herein that separate aliquots can include, for example, blood drawn from a subject into two separate vials, wherein each separate vial comprises a separate aliquot. In other embodiments, a biological sample is enriched for a given cell type prior to analysis in the assays described herein, thus the separate aliquots can be sub-samples of the enriched population of cells. Each of a plurality of aliquots is generally, but not necessarily, the same as the others, e.g., in terms of volume, approximate cell number, etc.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein. Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A human subject can be genetically male or female and of any age (e.g., neonate, infant, baby, toddler, child, pre-teen, adolescent, adult, geriatric etc).

A subject can be one who has been previously diagnosed with or identified as suffering from or having a condition in need of treatment or one or more complications related to a cancer, and optionally, have already undergone treatment for the cancer or the one or more complications related to the cancer.

As used herein, an “appropriate negative control” refers to an untreated, substantially identical cell or population that is treated in the same manner as the biological samples from a subject (e.g., in the same assay “run” simultaneously or near-simultaneously) but the negative control is not treated with an anti-cancer agent. Thus, the negative control can represent the “maximal” degree of cell viability of the sample in the assay to which the treated cells/population can be compared.

As used herein, an “appropriate positive control” refers to a substantially similar cell or population that is tested within the same assay “run” (e.g., simultaneously or near-simultaneously) as the biological sample but comprises treatment of the sample with an agent known to cause cell death or a given degree of cell death. A positive control can serve as an indicator that the assay run was successful. A positive control can be identified by a measurable reduction in e.g., partial or complete loss of cell viability. While positive controls will vary, e.g., with different cancer or cancer cell types. In one embodiment, the positive control for cancer cells (e.g., blast cells from a AML patient) comprises mitomycin C.

As used herein, the term “personalized treatment” refers to a given anti-cancer agent or combination of anti-cancer agents that have shown effectiveness in killing cancer cells (e.g., cancer stem cells and/or non-stem cell cancer cells) present in a subject's biological sample. That is, a treatment is selected for a subject based on the performance of a given drug(s) in a functional assay from the subject (i.e., tailored to a subject or to the subject's cancer).

As used herein the term “effective to kill CSCs or NSCCCs” means that cell viability of a patient sample is reduced by at least 20% in the presence of an agent as compared to an appropriate negative control (e.g., untreated sample). In other embodiment, the cell viability of a sample in the presence of an agent is reduced by at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 95%, at least 98% or even 100% (i.e., complete loss of cell viability in the sample; below detection limit of a given cell viability assay)

As used herein, the phrase “on the basis of” refers to selection of an anti-cancer agent that is based, at least in part, on the measured cell viability or other output or parameter derived from the data of a functional cell assay as described herein. It will readily be recognized that, in a clinical setting, there are other factors that can be taken into account when selecting an anti-cancer agent or combination of agents in the treatment of a given subject. Such factors can include age, underlying disease or illness directly related to the cancer, presence of other diseases or disorders that may not be directly related to the cancer (e.g., heart disease, kidney disease etc.), general health of the subject, insurance coverage, accessibility, patient compliance, allergy, mutations, side effects etc. However, it will be understood that the decision regarding which agent to select for treatment of the subject will depend, in part, on the results of the functional cell assay described herein. For example, when the therapeutic agents are ranked in order of efficacy one can select the top-ranked agent (i.e., highest rate of cell killing), however if that agent is contraindicated for any reason, a different agent is selected from the ranked list.

In certain embodiments, an anti-cancer agent is selected based on the EC50 concentration (i.e., the amount of the agent necessary to kill 50% of cells) of the agent in the functional assay as described herein. In some embodiments, cancer cells are determined to be susceptible to a given anti-cancer agent when the EC50 value for that anti-cancer agent is at least ten-fold lower than the maximal plasma concentration of that same anti-cancer drug that is achieved in humans (i.e., at least 20-fold lower, at least 30-fold lower, at least 40-fold lower, at least 50-fold lower, at least 60-fold lower, at least 70-fold lower, at least 80-fold lower, at least 90-fold lower, at least 100-fold lower or more). Thus, at clinically relevant doses (i.e., within the therapeutic window) of the given anti-cancer agent, one would expect to see greater than 50% cell death of cancer cells or cancer stem cells in a subject (i.e., greater than the EC50 value); when the EC50 is ten to 100-fold lower than the maximal plasma concentration, it is expected that substantially all of the cancer cells can be killed using a clinically relevant concentration of the anti-cancer agent. In one embodiment, the EC50 of a given anti-cancer agent selected for therapy as described herein is ten to 100-fold lower than the maximal plasma concentration for that anti-cancer agent in a subject.

As used herein, the term “percent cell death” refers to a relative degree of cell death in an aliquot treated with an agent or combination of agents as compared to the total number of cells in an untreated aliquot, which is representative of the maximal number of alive cells. As will understood by those of skill in the art, the relative number of living cells can be calculated by taking the ratio of: (# living cells in treated aliquot)/(# living cells in untreated aliquot)×100. The percent cell death is represented by: 100-% living cells. Ideally and in particular for the treatment of cancer stem cells, a therapeutic agent administered as a monotherapy will cause cell death of substantially all of the cancer stem cells (i.e., 100%, or at least 99%, or at least 98%, or at least 95% of cells). It is specifically contemplated herein that at least two therapeutic agents each with lower rates of cell killing or percent cell death (i.e., 90% or less, 80% or less, 70% or less, 60% or less, 50% or less, 40% or less, 30% or less or 20% or less) can be combined to achieve higher rates of cell death that can be used therapeutically with the aim of killing all of the detectable cells in the aliquot or subject. In some embodiments, such combination therapies can comprise at least two agents from at least two different classes that have complementary but not identical mechanisms of action.

As used herein, the terms “therapies” and “therapy” can refer to any method(s), composition(s), and/or agent(s) that can be used in the prevention, treatment and/or management of a cancer or one or more symptoms thereof. In certain embodiments, the terms “therapy” and “therapies” refer to chemotherapy, small molecule therapy, radioimmunotherapy, toxin therapy, prodrug-activating enzyme therapy, biologic therapy, antibody therapy, surgical therapy, hormone therapy, immunotherapy, anti-angiogenic therapy, targeted therapy, epigenetic therapy, demethylation therapy, histone deacetylase inhibitor therapy, differentiation therapy, radiation therapy, or a combination of the foregoing and/or other therapies useful in the prevention, management and/or treatment of a cancer or one or more symptoms thereof.

As used herein, the term “effective amount” refers to the amount of a therapy that is sufficient to reduce the severity or duration of cancer, ameliorate one or more symptoms of cancer, prevent the advancement of cancer, cause regression of cancer, result in the prevention of the development, recurrence, or onset of cancer and one or more symptoms thereof, to enhance or improve the prophylactic effect(s) of another therapy, and/or enhance or improve the therapeutic effect(s) of another therapy. In an embodiment of the invention, the amount of a therapy is effective to achieve one, two, three, or more results following the administration of one, two, three or more therapies: (1) a stabilization, reduction or elimination of the cancer stem cell population; (2) a stabilization, reduction or elimination in the non-stem cell cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, entry of the subject into remission; (9) a decrease in hospitalization rate, (10) a decrease in number of hospitalizations or lengths of stay, (11) the size of the tumor is maintained and does not increase or increases by less than 10%, preferably less than 5%, preferably less than 4%, preferably less than 2%, (12) an increase in the number of patients in remission, (13) an increase in the length or duration of remission, (14) a decrease in the recurrence rate of cancer, (15) an increase in the time to recurrence of cancer, and (16) an amelioration of cancer-related symptoms and/or quality of life.

As used herein, the term “in combination” in the context of the administration of a therapy to a subject refers to the use of more than one therapy (e.g., prophylactic and/or therapeutic). The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, the terms “manage,” “managing,” and “management” in the context of the administration of a therapy to a subject refer to the beneficial effects that a subject derives from a therapy (e.g., a prophylactic or therapeutic agent) or a combination of therapies, while not resulting in a cure of cancer. In certain embodiments, a subject is administered one or more therapies (e.g., one or more prophylactic or therapeutic agents) to “manage” cancer so as to prevent or limit the progression or worsening of the condition.

As used herein, the term “refractory” is most often determined by failure to reach a clinical endpoint, e.g., response, extended duration of response, extended disease free survival, relapse free survival, progression free survival and overall survival. Another way to define being refractory to a therapy is that a patient has failed to achieve a response to a therapy such that the therapy is determined to not be therapeutically effective. In some embodiments, a subject's cancer that is “refractory” displays resistance to a given anti-cancer agent or class of anti-cancer agents.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” refers to compounds and compositions which may be administered to mammals without undue toxicity. The term “pharmaceutically acceptable carriers” excludes tissue culture medium. Exemplary pharmaceutically acceptable salts include but are not limited to mineral acid salts such as hydrochlorides, hydrobromides, phosphates, sulfates, and the like, and the salts of organic acids such as acetates, propionates, malonates, benzoates, and the like.

As used herein, the term “comprising” means that other elements (e.g., including other elements with a similar action) can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention.

### Cancers

Provided herein are methods and assays for selecting one or more anti-cancer agents for treatment of a subject having cancer, based on a functional cellular assay using a biological sample obtained from the subject. In some embodiments, the subject is being treated for a cancer. Alternatively, the subject has been diagnosed with a cancer but is treatment naive with respect to anti-cancer therapies.

A skilled clinician can diagnose a subject as having cancer using any cancer screening methods known in the art and including, but not limited to, physical examination (e.g., prostate examination, rectal examination, breast examination, lymph nodes examination, abdominal examination, skin surveillance, testicular exam, general palpation), visual methods (e.g., colonoscopy, bronchoscopy, endoscopy), PAP smear analyses (cervical cancer), stool guaiac analyses, blood tests (e.g., complete blood count (CBC) test, prostate specific antigen (PSA) test, carcinoembryonic antigen (CEA) test, cancer antigen (CA)-125 test, alpha-fetoprotein (AFP), liver function tests), karyotyping analyses, bone marrow analyses (e.g., in cases of hematological malignancies), histology, cytology, flow cytometry, a sputum analysis and imaging methods (e.g., computed tomography (CT), magnetic resonance imaging (MRI), ultrasound, X-ray imaging, mammography, PET scans, bone scans).

The methods and assays described herein can be applied to any cancer, particularly those cancers that comprise cancer stem cells. Non-limiting examples of cancers include: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome (MDS); chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, non-secretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor and acromegaly; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor: esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendothcliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Other cancers or abnormal proliferative diseases, include but are not limited to, the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma, rhabdomyoscarcoma and osteosarcoma; other tumors, including melanoma, seminoma, tetratocarcinoma, neuroblastoma and glioma; tumors of the central and peripheral nervous system, including astrocytoma, neuroblastoma, glioma, and schwannomas; and other tumors, including melanoma, xeroderma pigmentosum, keratoactanthoma, seminoma, thyroid follicular cancer and teratocarcinoma. Cancers associated with aberrations in apoptosis are also included and are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, the cancer or abnormal proliferative disease comprises a malignancy or dysproliferative changes (such as metaplasias and dysplasias), or hyperproliferative disorders of the skin, lung, liver, bone, brain, stomach, colon, breast, prostate, bladder, kidney, pancreas, ovary, and/or uterus.

In some embodiments, the carcinoma or sarcoma includes, but is not limited to, carcinomas and sarcomas found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. The types of carcinomas include but are not limited to papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma. The types of sarcomas include but are not limited to, for example, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

In one embodiment, the methods and assays provided herein are used to inform treatment of a subject having leukemia. Non-limiting examples of leukemias and other blood-borne cancers include acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, and hairy cell leukemia.

In one embodiment of the methods, the subject having the tumor, cancer or malignant condition is undergoing, or has undergone, treatment with an anti-cancer therapy. In some embodiments, the cancer therapy is chemotherapy, radiation therapy, immunotherapy or a combination thereof.

### Biological Samples

Essentially any biological sample comprising cancer stem cells can be used with the methods and assays provided herein. Such samples can be obtained from a variety of sources, including blood and tumor sites. In some embodiments, samples such as bone marrow (e.g., a bone marrow aspiration), plasma, whole blood, red blood cell-depleted blood, cerebrospinal fluid, pleural fluid, pericardial fluid, ascites fluid, urine, lymph, synovial fluid, or isolated cells thereof and the like can be used with the methods and assays described herein. An appropriate biological sample comprising CSCs or CSCs and NSCCCs to test for susceptibility of a cancer to a panel of anti-cancer agents will be readily apparent to those of skill in the art.

It is emphasized that while some embodiments can incorporate one or more separation or fractionation steps performed on a sample, this is not necessarily required of other embodiments. Where, for example, markers for CSCs and NSCCCs can be measured simultaneously in a sample comprising both cancer cell types, the methods described herein can be performed without a sample fractionation or cell separation step or steps. In other embodiments, fractionation or enrichment can provide benefit, e.g., by increasing the sensitivity of detection of anti-cancer stem cell efficacy.

A biological sample can be a sample that has been treated in some manner, for example, separated by centrifugation, elutriation, density gradient separation, apheresis, affinity selection, panning, or fluorescence assisted cell sorting (FACS). In some embodiments, the biological sample has been subjected to one or more pretreatment steps prior to its use in the methods and assays described herein. In certain embodiments, a biological fluid is pretreated by centrifugation, filtration, precipitation, dialysis, or chromatography, or by a combination of such pretreatment steps. In other embodiments, a tissue sample is pretreated by freezing, chemical fixation, paraffin embedding, dehydration, permeablization, or homogenization followed by centrifugation, filtration, precipitation, dialysis, enrichment of a desired cell type, or chromatography, or by a combination of these pretreatment steps.

The sample can be obtained by any convenient procedure, such as the drawing of blood, venipuncture, biopsy, or the like. In some embodiments, a sample will comprise at least about 10² cells, at least about 10³ cells, at least about 10⁴ cells, at least about 10⁵ cells or more. Typically, the samples will be from human patients, although animal models may find use, e.g. equine, bovine, porcine, canine, feline, rodent, e.g. mice, rats, hamster, primate, etc. The subject from which a sample is obtained and utilized in accordance with the methods described herein includes, without limitation, an asymptomatic subject, a subject manifesting or exhibiting 1, 2, 3, 4 or more symptoms of cancer, a subject clinically diagnosed as having cancer, a subject predisposed to cancer metastases, a subject that previously underwent treatment for a cancer, a subject undergoing therapy for cancer, a subject that has been medically determined to be free of cancer (e.g., following therapy for the cancer), or a subject that is managing cancer. In certain embodiments, the term “has no detectable cancer,” as used herein, refers to a subject or subjects in which there is no detectable cancer by conventional methods, e.g., MRI. Once a sample is obtained, it can be used directly, frozen, or maintained in appropriate culture medium for short periods of time. Various media can be employed to maintain cells.

In some embodiments, a control biological sample can be tested alongside the biological sample in the assays described herein. Such samples can be used to normalize cell viability results and/or serve as a comparison to assess efficacy of a given agent. Control biological samples can be a reference sample taken from the subject prior to the administration of cancer therapy or is a sample taken from the subject one week, two weeks, one month, two months, three months, six months, or one year prior to administration of the therapy. In other embodiments, the control biological sample or reference sample is taken from the subject during, or following the administration of a given anti-cancer therapy. For example, the reference sample may be taken from the subject one week, one month, two months, three months, six months, or one year following administration of the therapy. In other aspects, a reference sample can be a sample from a patient or a population of patients in remission from the same cancer or a sample from a healthy patient with no detectable cancer or a population of healthy patients with no detectable cancer. In certain embodiments, a positive or negative control sample is a sample that is obtained or derived from a corresponding tissue or biological fluid or tumor as the sample to be analyzed in accordance with the methods as described herein. This sample will typically be from the same patient at the same or different time points.

In certain embodiments, the biological sample is blood, urine, bone marrow or interstitial fluid. In another embodiment, the sample is a tissue sample, such as a tissue sample from breast, brain, skin, colon, lung, liver, ovary, pancreas, prostate, kidney, bone or skin tissue. In one embodiment, the tissue sample is a biopsy of normal and/or tumor tissue. The amount of biological sample taken from the subject will vary according to the type of biological sample and the method of detection to be employed. In some embodiments, the amount of blood, urine, or bone marrow taken from the subject is 0.1 ml, 0.5 ml, 1 ml, 5 ml, 8 ml, 10 ml or more. In another embodiment, the amount of cancer or tumor tissue taken from the subject is less than 10 milligrams, less than 25 milligrams, less than 50 milligrams, less than 1 gram, less than 5 grams, less than 10 grams, less than 50 grams, or less than 100 grams.

### Cancer Stem Cells

Cancer stem cells comprise a unique subpopulation (often 0.1-10% or so) of a tumor that, relative to the remaining 90% or so of the tumor (i.e., the tumor bulk), are more tumorigenic, relatively more slow-growing or quiescent, and often relatively more chemoresistant than the tumor bulk. Given that conventional therapies and regimens have, in large part, been designed to attack rapidly proliferating cells (i.e. those cancer cells that comprise the tumor bulk; “acute” cancer), cancer stem cells which are often slow-growing can be relatively more resistant than faster growing tumor bulk to conventional therapies and regimens. Cancer stem cells can express other features which make them relatively chemoresistant such as multi-drug resistance, anti-apoptotic pathways, sheltered location, and reduced uptake of anti-cancer agents or drugs. The aforementioned would constitute a key reason for the failure of standard oncology treatment regimens to ensure long-term benefit in most patients with advanced stage cancers—i.e. the failure to adequately target and eradicate cancer stem cells. In some instances, a cancer stem cell(s) is the founder cell of a tumor (i.e., it is the progenitor of the cancer cells that comprise the tumor bulk).

Cancer stem cells have been identified in many different cancer types and comprise a given marker profile. Such marker profiles can permit detection and, when combined with a cell viability assay, viability measurement for CSCs. Such profiles can optionally be used to generate a population enriched for CSCs. For example, leukemia stem cells can be identified using the marker phenotype CD34+ CD38− (see e.g., Bonnet et al., Nat Med 3:730-737 (1997)). In addition, human acute lymphoblastic leukemia (ALL) cells can comprise the marker phenotype: CD34*/CD10− or CD34−/CD19−. (See e.g., Cox et al., Blood 104(19): 2919-2925 (2004). Additional representative cancer stem cell marker profiles are shown in the following Tables.

TABLE 1
Marker Phenotype/Profiles for Exemplary Cancer Stem Cells

| Cancer Type | Marker Phenotype/Profile | Reference(s) |
|---|---|---|
| Multiple myeloma | CD138- | Matsui et al. (2004) Blood 103(6): 2332 |
| Breast Cancer | 1. CD44+CD24low lin- | 1. Al-Hajj et al., Proc. Natl. Acad. Sci. |
| | 2. CD44+, CD24−, ALDH1+ | USA 100:3983-3988 (2003) |
| | TSPAN8 | 2. Carrasco et al. European Journal of |
| | | Clinical Investigation 44(7):678-687 |
| Prostate Cancer | CD44+/alpha2beta1hi/CD133+; | Collins et al., Cancer Res 65(23):10946- |
| | CD44+/CD24−, CD133+, | 10951 (2005); Sharpe et al. Stem Cell |
| | α2β1^{high}, ALDH1+ | Reviews 9(5): 721-730 (2019) |
| Melanoma | CD20+, CD133+, CD271+ | Fang et al., Cancer Res 65(20): 9328- |
| | | 9337 (2005); Lang et al., Clinics in |
| | | Dermatology 31(2):166-178 |
| Brain Tumors | CD133+, CD44+ | Singh et al., Nature 432:396-401 (2004); |
| | | Singh et al., Oncogene 23:7267-7273 |
| | | (2004); Singh et al., Cancer Res. |
| | | 63:5821-5828 (2003). Jackson et al., |
| | | Carcinogenesis 36(2):177-185 |
| Colon Cancer | CD44+, CD133+, CD166+, | Botchkina et al., Cancer Letters |
| | CD24+, EpCAM+, ESA+, ALDH1+ | 338(1):127-140 (2013); |
| | | Tseng et al. Cancer Cell 8(4):3223-335 |
| | | (2015) |
| Esophagus | CD44+, CD24+, CD133+, | Qian et al. Onco Targets and Therapy |
| | ABCG2+, CXCR4+, ALDH1+ | 9:22247-2254 (2016) |
| Stomach cancer | CD44+, CD44V8-10+, CD133+, | Brungs et al. Journal of |
| | CD24+, CD54+, CD90+, CD49f+, | Gastroenterology 51(4): 313-326 (2016) |
| | CD71+, EpCAM+, ALDH1+ | |
| Pancreatic cancer | CD44+/CD24+, CD133+, ESA-i-, | Li et al., Cancer Research 67(3):1030- |
| | ALHD1+ | 1037 (2007); Zhan et al., Cancer Letters |
| | | 357(2):429-437 (2015) |
| Lung cancer | CD44+, CD133+, CD166+, | Lundin and Driscoll. Cancer Letters |
| | ALDH1+ | 338(1): 89-93 (2013) |
| Ovarian Cancer | CD44+, CD133+, CD24+, | Zhan et al., BioMed Research |
| | CD117+, EpCAM+, ALDH+ | International 916819 (2013) |
| Liver cancer | CD44+, CD133+, CD90+, CD13+, | Sun et al., World Journal of |
| | EpCam+ | Gastroenterology 22(13):3547-3557 |
| | | (2016) |
| Head & Neck squamous | CD44+, CD133+, ALDH+, BMI1+, | Krishnamurthy et al., Journal of Dental |
| cell carcinoma | Sox2+, Oct4+, ABCB5+, AGR2+, | Research 91(4):334-340 (2012) |
| | Taz+ | |
| AML | CD34+/−, CD38+/−, CD90+/−, | Horton and Huntly. Haematologica |
| | CD123+, CD45RA+, CD33+, | 97(7):966-974 (2012) |
| | CD13+, CD44+, CD96+, CD47+, | |
| | CD32+, CD25+, CLL-1+, TIM3+ | |
| Multiple myeloma | CD138−, CD19+, CD27+ | Matsui et al., Cancer Research |
| | | 68(1):190-197 (2008) |

TABLE 2
Additional or alternative Marker Phenotype/Profiles for Cancer Stem Cells

| Cancer Type | Marker Phenotype/Profile |
|---|---|
| Leukemia (AML)/ | CD34+/CD38− |
| Chronic Myelogenous | |
| Leukemia (CML) | |
| Leukemia (ALL) | CD34+/CD10−/CD19− |
| Ovarian | CD44+/CD24− |
| Multiple Myeloma | CD138−/CD34−/CD19+ |
| Ependymoma | CD133+/RC2+ |

In some embodiments, a cancer stem cell marker or combination thereof is selected from those described in e.g., Abbaszadegan et al. Journal of Cellular Physiology 232:2008-2018 (2017); Lu, L et al. Medicine 95:42(e5163 (2016); Curtarelli, R et al. Stem Cell Reviews and Reports 14:769-784 (2018); Zhu, R et al. Nature Communications 10:2863 (2019); Yu, Z et al. Int J Biochem Cell Biol 44(12):2144-2151 (2012), the contents of each of which are incorporated herein by reference in their entirety. Additional cancer stem cell markers that can be used to identify or isolate CSCs for use with the methods and assays described herein include, but are not limited to, CD123, CLL-1, combinations of SLAMs (signaling lymphocyte activation molecule family receptors; see Yilmaz et al., Hematopoiesis 107: 924-930 (2006)), such as CD150, CD244, and CD48, and those markers disclosed in U.S. Pat. No. 6,004,528, the contents of which are incorporated herein by reference in its entirety.

In addition, suitable cancer stem cell antigens to identify or isolate CSCs for use with the methods described herein can be identified: (i) through publicly available information, such as published and unpublished expression profiles including cell surface antigens of cancer stem cells of a particular tumor type or adult stem cells for a particular tissue type, and/or (ii) by cloning cancer stem cells or adult stem cells of a particular tumor or tissue type, respectively, in order to determine their expression profiles and complement of cell surface antigens. Cloning of normal stem cells is a technique routinely employed in the art (Uchida et al., Curr. Opin. Immunol, 5:177-184 (1993)).

In some embodiments, a population of cells enriched for CSCs comprises less than 20% non-stem cell cancer cells (NSCCCs). In other embodiments, the population of cells enriched for CSCs comprises less than 15%, less than 10%, less than 5%, less than 2%, less than 1% or even lacks all detectable NSCCCs as determined using standard methods known in the art.

Leukemic Stem Cells: Survival of leukemia initiating cells, also known as leukemia stem cells (LSCs), may play a critical role in relapse of patients after treatment of acute myeloid leukemia (AML), despite successful destruction of the bulk of the leukemic blasts. LSCs are defined classically by their capacity to engraft in immunodeficient mice, differentiate, proliferate, and renew. Unlike the non-clonogenic blasts they give rise to, LSCs are quiescent and protected in localized marrow endosteal niches like their normal hematopoietic stem cell (HSC) counterparts, rendering them less susceptible to cell cycle targeted chemotherapy. A “dormancy” gene signature has been associated with LSCs, with prominent expression of genes associated with adhesion within the marrow microenvironment (e.g., SPP1 (osteopontin), ITGA3 (integrin alpha 3), ITGAV (vitronectin receptor) and CD44). Most data support the reasoning that both in vivo (xenograft) and in vitro resistance to a given anti-cancer agent is due to the resistance or persistence of the LSC fraction in the presence of standard chemotherapy (Ishikawa et al. (2007) Nature Biotechnology 25:1315-1321). The clinical significance of LSCs is also supported by prognostic correlation. For example, in AML patients achieving morphologic complete remission (CR), the residual disease (MRD) is enriched for LSCs, and in newly diagnosed AML patients, a high proportion of LSCs predicts treatment failure and poor prognosis. Further, increased expression of genes proposed to be specific for the LSCs within patient samples correlates with treatment resistance and a decrease in overall survival, event free survival and relapse-free survival in AML. Characterization of LSCs has led to targeted drug development, but this principle has not yet changed clinical management of AML.

LSCs are heterogeneous, yet are most frequently enriched in the CD34+CD38lo or negCD123+ fraction. A neutralizing antibody to CD123 reduces engraftment potential and eliminates secondary transplants in NODscid mice, indicating that LSCs express CD123. These lineage markers identify leukemia-initiating clones but likely do not constitute all cells with engraftment potential in immunodeficient mice, as variability in CD38 expression has also been observed for normal cord blood progenitors that engraft NODscid mice.

Decades of work have established that AML is a heterogeneous malignancy with considerable cytogenetic and molecular variability between patients and also clonal diversity within the individual patient. However, AML resembles many other malignancies in being genetically unstable; at diagnosis, resistance to any single class of therapy is likely to be present in LSCs and additional resistant phenotypes are predicted to be acquired with tumor evolution. Simulations of this evolution underscore the importance of minor subclones in determining long-term outcomes. Mutation analysis in subclones of LSCs may play a role in predicting response. Recent work demonstrated that mutation patterns in human AML subclones were affected, presumably due to selection pressure, by their engraftment in NOD/SCID IL2R γc−/− and NOD/SCID IL2R γc−/−-SGM3 mice. Using AML as a proof-of-concept, the working Examples described herein use functional drug screening and mutation analysis before and after NOD/SCID IL2R γc−/− mouse engraftment to gain insight into an additional mutational basis for drug resistance in AML.

In some embodiments, the leukemia stem cells (LSCs) can be responsible for progression and drug resistance in leukemias. The LSCs described herein are shown to have the phenotype similar to that of a hematopoietic progenitor cell, but altered in that the cells have acquired the proliferative and self-renewal capacity that is normally restricted to hematopoietic stem cells.

With respect to myelogenous leukemias, e.g. CML, AML, etc., the phenotype of myeloid lineage progenitors is useful in identification and/or isolation of a population enriched in LSCs. These progenitor cells stain negatively for the markers Thy-1 (CD90), IL-7Ra (CD127); and with a panel of lineage markers, which lineage markers may include CD2; CD3; CD4; CD7; CD8; CD10; CD11b; CD14; CD19; CD20; CD56; and glycophorin A (GPA) in humans and CD2; CD3; CD4; CD8; CD19; IgM; Ter110; Gr-1 in mice. Typically, an LSC can be identified using the marker profile: CD34+CD38-.

The LSCs identified in CML (CML-LSC) can have a phenotype comprising: CD34+CD38+IL-3Rα+CD45RA+ and be negative for a panel of lineage markers, which may comprise CD2; CD3; CD4; CD7; CD8; CD10; CD11b; CD14; CD19; CD20; CD56; and glycophorin A (GPA). The cells can further be identified by their ability to self-renew in vitro; and the presence of an activated β-catenin pathway, which can be inhibited with axin.

Other LSC subsets that can find use in isolating and/or identifying LSCs for use with the methods described herein include the common lymphoid progenitor, e.g. in analysis of lymphocytic leukemias. Common lymphoid progenitors, CLP, express low levels of c-kit (CD117) on their cell surface. Antibodies that specifically bind c-kit in humans, mice, rats, etc. are known in the art. Alternatively, the c-kit ligand, steel factor (Slf) may be used to identify cells expressing c-kit. The CLP cells express high levels of the IL-7 receptor alpha chain (CDw127). Antibodies that bind to human or to mouse CDw127 are known in the art. Alternatively, the cells are identified by binding of the ligand to the receptor, IL-7. Human CLPs express low levels of CD34. Antibodies specific for human CD34 are commercially available and well known in the art. See, for example, Chen et al. (1997) Immunol Rev 157:41-51. Human CLP cells are also characterized as CD38 positive and CD10 positive. The CLP subset also has the phenotype of lacking expression of lineage specific markers, exemplified by B220, CD4, CD8, CD3, Gr-1 and Mac-1. The CLP cells are characterized as lacking expression of Thy-1 (CD90), a marker that is characteristic of hematopoietic stem cells. The phenotype of the CLP may be further characterized as Mel-14−, CD43lo, HSAlo, CD45+ and common cytokine receptor γ chain positive.

Megakaryocyte stem cells may also be used with the assays and methods described herein, for example with respect to megakaryocytic forms of AML. The MKP cells are positive for CD34 expression, and tetraspanin CD9 antigen. The MKP cells express CD41, also referred to as the glycoprotein IIb/IIIa integrin, which is the platelet receptor for fibrinogen and several other extracellular matrix molecules, for which antibodies are commercially available, for example from BD Biosciences, Pharmingen, San Diego, Calif., catalog number 340929, 555466. The MKP cells are positive for expression of CD117, which recognizes the receptor tyrosine kinase c-Kit. Antibodies are commercially available, for example from BD Biosciences, Pharmingen, San Diego, Calif., Cat. No. 340529. MKP cells are also lineage negative, and negative for expression of Thy-1 (CD90).

### Non-Stem Cell Cancer Cells (NSCCCs)

Non-stem cells cancer cells are cells associated with a tumor or cancer that do not display cancer stem cell character and can be separated by cancer stem cells on the basis of one or more markers (or lack of one of more stem cell markers). It will be appreciated by those of skill in the art that NSCCCs will lack one or more of the cancer stem cell markers listed in Tables 1 or 2. Treatment of NSCCCs is contemplated as treatment of “acute” cancer, while targeting of CSCs is contemplated as treatment of persistent cells that can cause relapse of the cancer.

In some embodiments, a population of cells enriched for NSCCCs comprises less than 20% cancer stem cells (CSCs). In other embodiments, the population of cells enriched for NSCCCs comprises less than 15%, less than 10%, less than 5%, less than 2%, less than 1% or even lacks detectable CSCs as determined using standard methods known in the art.

In some embodiments, an NSCCC lacks expression of a stem cell marker and/or comprises expression of one or more markers associated with the differentiated cell type to which the NSCCC corresponds.

### Anti-Cancer Agents

Any anti-cancer therapy which is useful, has been used, is currently being used, or may be used for the prevention, treatment and/or management of cancer can be used to prevent, treat, and/or manage the subject whose cancer stem cells are tested in accordance with the methods and assays described herein. Exemplary anti-cancer agents include, but are not limited to, peptides, polypeptides, fusion proteins, nucleic acid molecules, small molecules, mimetic agents, synthetic drugs, inorganic molecules, and organic molecules. Non-limiting examples of cancer therapies include chemotherapies, radiation therapies, hormonal therapies, anti-angiogenesis therapies, targeted therapies, and/or biological therapies including immunotherapies and surgery. In certain embodiments, a therapeutically effective regimen comprises the administration of a combination of at least two therapies or at least two agents.

Examples of anti-cancer therapies include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracyclin; anthramycin; asparaginase; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine (Ara-C); dacarbazine; dactinomycin; daunorubicin hydrochloride; decitabine (Dacogen); demethylation agents, dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; histone deacetylase inhibitors (HDAC-Is) hydroxyurea; idarubicin hydrochloride; ifosfamide; ilmofosine; imatinib mesylate (Gleevec, Glivec); interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; lenalidomide (Revlimid); letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper, mitotane; mitoxantrone hydrochloride; mycophenolic acid; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate: triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Additional exemplary anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TIC antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor, cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanrcotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor, mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor, multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; oracin; oral cytokine inducer, ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; gamma secretase inhibitors, single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; S-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; anti-integrin antibodies (e.g., anti-integrin αvβ3 antibodies); vorozole; zanoterone; zeniplatin; zilascorb; and zinostatin stimalamer.

In certain embodiments, anti-cancer agents that can be tested for efficacy using the methods and assays described herein is an alkylating agent, a nitrosourea, an antimetabolite, and anthracycline, a topoisomerase II inhibitor, or a mitotic inhibitor. Alkylating agents include, but are not limited to, busulfan, cisplatin, carboplatin, cholorambucil, cyclophosphamide, ifosfamide, decarbazine, mechlorethamine, mephalen, and themozolomide. Nitrosoureas include, but are not limited to carmustine (BCNU) and lomustine (CCNU). Antimetabolites include but are not limited to 5-fluorouracil, capecitabine, methotrexate, gemcitabine, cytarabine, and fludarabine. Anthracyclins include but are not limited to daunorubicin, doxorubicin, epirubicin, idarubicin, and mitoxantrone. Topoisomerase II inhibitors include, but are not limited to, topotecan, irinotecan, etopiside (VP-16), and teniposide. Mitotic inhibitors include, but are not limited to taxanes (paclitaxel, docetaxel), and the vinca alkaloids (vinblastine, vincristine, and vinorelbine).

Panel of anti-cancer agents: A panel of anti-cancer agents comprises at least two anti-cancer agents, preferably at least two anti-cancer agents or drugs, and depending upon testing format, most often less than 1000 different anti-cancer agents. Thus, in some embodiments, the panel of anti-cancer agents comprises at least 3, at least 4, at least 5, at least 6, at least 7, at least 8, at least 9, at least 10, at least 12, at least 15, at least 20, at least 25, at least 30, at least 35, at least 40, at least 45, at least 50, at least 55, at least 60, at least 65, at least 70, at least 75, at least 80, at least 85, at least 90, at least 95, at least 96, at least 100, at least 110, at least 120, at least 130, at least 140, at least 150, at least 160, at least 170, at least 180, at least 190, at least 200, at least 225, at least 250, at least 275, at least 300, at least 350, at least 400, at least 450, at least 475, at least 500, at least 550, at least 600, at least 650, at least 700, at least 750, at least 850, at least 900, or at least 950 anti-cancer agents or more, including any integer therebetween. In some embodiments, the panel of anti-cancer agents comprises at least 1000, at least 1500, at least 2000, at least 2500 or more agents.

In other embodiments, a panel of anti-cancer agents or drugs comprises 2-55, 2-400, 2-300, 2-200, 2-100, 2-50, 2-25, 2-5, 2-10, 2-5, 5-10, 5-25, 5-50, 5-100, 5-200, 5-300, 5-400, 5-500, 10-25, 10-50, 10-100, 10-200, 10-300, 10-400, 10-500, 25-50, 25-100, 25-200, 25-300, 25-400, 25-500, 50-75, 50-100, 50-200, 50-300, 50-400, 50-500, 100-200, 100-300, 100-400, 100-500, 200-300, 200-400, 200-500, 300-400, 300-500, 400-450, 400-500, or 440-500 different anti-cancer agents or drugs or any range therebetween. In other embodiments, a panel of anti-cancer agents comprises 2-500, 2-600, 2-700, 2-800, 2-900, 2-1000, 5-500, 5-600, 5-700, 5-800, 5-900, 5-1000, 10-500, 10-600, 10-700, 10-800, 10-900, 10-1000, 25-500, 25-600, 25-700, 25-800, 25-900, 25-1000, 50-500, 50-600, 50-700, 50-800, 50-900, 50-1000, 100-500, 100-600, 100-700, 100-800, 100-900, 100-1000, 200-500, 200-600, 200-700, 200-800, 200-900, 200-1000, 300-500, 300-600, 300-700, 300-800, 300-900, 300-1000, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, or 900-1000 anti-cancer agents or drugs are any range therebetween.

In some embodiments, the panel of anti-cancer agents comprises at least one drug from at least 2 different classes of drugs (e.g., antimetabolites, protease inhibitors, enzyme inhibitors etc).

In some embodiments, the panel of anti-cancer agents comprise tyrosine kinase inhibitors, histone deacetylase inhibitors, cyclin-dependent kinase inhibitors, proteasome inhibitors, targeted inhibitor, PARP inhibitors, alkylating agents, cisplatinum compounds, anthracyclines, topoisomerase inhibitors, Flt-3 kinase inhibitors, and microtubule assembly inhibitors.

### Assessing Cell Viability

The methods and assays described herein are not limited by the means by which cell viability is measured. That is, essentially any method can be used to measure the degree of cell viability in the methods and assays described herein. It will be appreciated by those of skill in the art that a suitable cell viability assay used with the assays and methods described herein will permit high-throughput analysis of a plurality of samples.

A variety of means and methods are known in the art for determining cell viability. For example, some methods are based on the ability of the membrane of viable cells to exclude vital dyes such as trypan blue, propidium iodide, and ethidium monoazide. Living cells exclude such vital dyes whereas dead or dying cells that have lost membrane integrity permit entry of these dyes into the cytoplasm, where the dyes stain various compounds or organelles within the cell. Non-viable cells that have lost membrane integrity also leak cytoplasmic components into the surrounding medium. Cell death thus can be measured by monitoring the concentration of these cellular components in the surrounding medium. For example, the release of glyceraldehyde-3-phosphate dehydrogenase (G3PDH) from dead or damaged cells is measured by coupling the activity of the released G3PDH to the production of ATP (Corey et al. (1997) J. Immunol. Meth. 207:43-51).

Other methods to test for cell viability/cell death rely upon the conversion of a dye from one state to another. For example, in a typical format, prior to the reaction the dye absorbs at a first wavelength of radiation. The dye is then converted to a product that absorbs at a second (and different) wavelength of light. By monitoring the conversion of the dye from one state to the other, the extent of cell viability or cell death can be determined. A number of suitable dyes for this purpose are known, and of these indicators, electron-acceptor dyes such as tetrazolium salts are frequently used. Tetrazolium salts known in the art include MTT (3-(4,5-dimethylthiazol-2-yl)-2,5-diphenyltetrazolium bromide), XTT (sodium 3′-(1-phenylamino-carbonyl)-3,4-tetrazolium-bis(4-methoxy-6-nit-ro)benzene-sulfonic acid hydrate), and MTS (3-(4,5-dimethylthiazol-2-yl)-5-(3-carboxymethoxyphenyl)-2-(4-sulphophenyl)-2H-tetrazolium, inner salt). See, for example, van de Loosdrecht et al. (1994) J. Immunol. Methods 174:311-320; Buttke et. al. (1993) J. Immunol. Methods 157: 233-240; Berridge et al. (2005) Biotechnol. Annu. Rev. 11:127-152.

As a specific non-limiting example, resazurin is a non-toxic, cell permeable compound that, in its oxidized state, is blue in color and virtually non-fluorescent. In living cells, resazurin is reduced to resorufin, a compound that is red in color and highly fluorescent, and can be detected fluorimetrically or colorimetrically. Metabolic activity of viable cells continuously converts resazurin to resorufin, increasing the overall fluorescence and color of the media surrounding cells. O'Brien et al. (2000) Eur. J. Biochem. 267:5421-5426.

Cell viability and cytotoxicity assays based on detection of release of LDH by dead cells are also commercially available. For example, Promega Corporation markets CytoTox 96® Assay, Sigma-Aldrich markets Tox-7 In Vitro Toxicology Assay Kit, and G-Biosciences markets CytoScan™ LDH Cytotoxicity Assay.

Additional exemplary assays include a tetrazolium reduction assay (i.e., MTT, MTS, XTT, and WST-1 assays), a resazurin assay, a protease viability marker assay, an ATP assay, DNA condensation assays (e.g., Hoechst 33258, Acridine Orange, and the like), and annexin V assay, and a real-time assay for viable cells (see e.g., Riss et al. (2013) “Cell Viability Assays” in Sittampalam, G S et al., editors Assay Guidance Manual Bethesda, Md.: Eli Lilly & Company and the National Center for Advancing Translational Sciences, 2004-, the contents of which are incorporated herein by reference in their entirety). Reagents to assess cell viability can be obtained from a variety of commercial sources including, but not limited to, Promega (Madison, Wis.), Thermo Fisher Scientific (Waltham, Mass.), Cell Biolabs, Inc. (San Diego, Calif.), Molecular Devices (San Jose, Calif.), Cayman Chemical (Denver, Colo.), among others.

Flow cytometry is a well-known method for identifying and distinguishing between different cell phenotypes in a non-homogeneous sample and can be used to assess cell viability of a sample quantitatively (see e.g., Kummrow et al. (2013) Cytometry Part A 83A:197-204). In the cytometer, cells are passed through a cuvette flow cell where they are beamed by one or more energy sources. In the sensing regions, light which is scattered or emitted by the cells is detected. In some embodiments, the flow cytometry sample for use with the methods and assays described herein comprises an enriched or purified population of cancer stem cells or non-stem cell cancer cells.

In addition, high-content methods are contemplated herein for assessing cell viability (see e.g., US2018/0043357, US2018/0328914 and US2018/003613, the contents of each of which are incorporated herein by reference in their entirety). As used herein, the term “high-content assay” refers to a phenotypic assay conducted on cells that can measure a plurality (e.g., at least two) different parameters (e.g., cell viability, metabolic phenotype, expression of cell surface marker etc.) from the same sample. In some embodiments, the high-content assay comprises simultaneous or near simultaneous measurement of a plurality of different parameters. In some embodiments, the high-content method comprises continuous, multiplexed readouts. In some embodiments, a high-content method comprises a microfluidic device. In one embodiment, one of the phenotypic parameters permits differential detection of cancer stem cells and/or non-stem cell cancer cells.

### High Throughput Screening

High throughput screening (HTS) assays and techniques of various types are typically used to screen chemical libraries consisting of large numbers of small molecules for their ability to suppress or enhance disease processes. Cell-based assays in a high throughput format can provide information on possible resistance or susceptibility of cells to a given anti-cancer agent.

Automated HTS assays and techniques and robotic systems for drug discovery have been described. The ability to perform a wide variety of biochemical and molecular biology tests using automated systems is widely known, including the ability to perform tests based using enzymatic activity, ELISA, receptor binding, macromolecular interactions, protein expression, and protein folding and assembly. Screens can be carried out using multi-well microtiter plates. In certain embodiments, the high throughput screening assays used herein comprise fluorescence assisted cell sorting (FACS).

One of skill in the art will understand the benefits of high-throughput assays and can scale up a given cell viability assay to a high-throughput assay. Given that HTS methods are known to those of skill in the art, such methods are not described in detail herein.

The output or read-out of the functional cell assay as described herein can be determined qualitatively, for example, by assessing the relative reduction in cell viability in the presence of an anti-cancer agent to which the sample is sensitive to as compared to an untreated substantially similar sample, or by ranking the individual members of the panel of anti-cancer agents in functional order (e.g., most effective to least effective or vice versa). The read-out of a panel of anti-cancer agents can be measured quantitatively using any of a variety of analyses, such as, specificity, sensitivity, positive predictive value, negative predictive value, diagnostic accuracy, or AUC.

A functional cell assay as described herein can be used with a variety of different assay formats. For example, chip based assays or microtiter assays enable simultaneous testing of multiple anti-cancer agents. Thus, a number of controls, positive and negative, can be included in the assay. The assay then can be run with simultaneous treatment of plural samples from a given subject (i.e., separate aliquots), such as multiple samples to be tested with a panel of anti-cancer agents and optionally different concentrations of each individual member of the panel of anti-cancer agents, a negative control sample, a positive control sample, a blank, and so on. Including internal controls in the assay allows for normalization, calibration and standardization of signal strength within the assay. For example, each of the positive controls, and negative controls can be run in plural, and the plural samples can be a serial dilution. The control samples can be randomly arranged among the test samples to minimize variation due to sample site location on the testing device.

Thus, such high throughput assays comprising internal controls enable one to test the sensitivity of a plurality of separate aliquots from a subject to one or more therapeutics by simultaneous or near-simultaneous measurement. An exemplary embodiment of the high-throughput functional cell assays contemplated herein is described in detail in the working Examples.

In some embodiments, the functional cell assays described herein further comprise quality control metrics (QC). QC metrics can be evaluated with the help of QC markers that provide information indicative of one or more category of information. In some embodiments, a QC marker is indicative of duration of sample storage, maximum temperature exposure, minimum temperature exposure, average temperature exposure, time-temperature exposure, sample pH, light exposure, UV exposure, radiation exposure, humidity, elution efficiency of sample constituents, hydropathy-associated elution efficiency, overall sample elution efficiency, sample stability, proteolytic activity, DNase activity, or RNase activity. Non-limiting examples of QC markers include elution markers, humidity markers, pH markers, temperature markers, time markers, proteolysis markers, nuclease markers, stability markers, radiation markers, UV markers, and light markers.

### Dosage and Administration

In some aspects, the methods described herein provide a method for treating cancer in a subject by administering an anti-cancer agent selected using the methods and assays described herein. In one embodiment of this aspect and all other aspects described herein, the cancer is leukemia (e.g., AML). In one embodiment, the subject can be a mammal. In another embodiment, the mammal can be a human, although the approach is effective with respect to all mammals. The methods comprise administering to the subject an effective amount of a pharmaceutical composition comprising an anti-cancer agent or combination of anti-cancer agents determined to be effective in a given subject based on the cell viability measures employed in the methods and assays described herein. The appropriate dosage range for a given anti-cancer agent depends upon the potency, and includes amounts large enough to produce the desired effect, e.g., treatment of cancer or reduction in number of CSCs. Although adverse side effects are often associated with anti-cancer agents, the dosage should not be so large as to cause unacceptable or life-threatening adverse side effects. Generally, the dosage will vary with the type of inhibitor, and with the age, condition, and sex of the patient. The dosage can be determined by one of skill in the art and can also be adjusted by the individual physician in the event of any complication.

Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Currently available anti-cancer therapies and their dosages, routes of administration and recommended usage are known in the art and have been described in such literature as the Physician's Desk Reference (60th ed., 2017).

Administration of the doses recited above or as employed by a skilled clinician can be repeated for a limited and defined period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and continued responsiveness to therapy. Continuous, relatively low maintenance doses are contemplated after an initial higher therapeutic dose.

A therapeutically effective amount is an amount of an agent that is sufficient to produce a statistically significant, measurable change of a given symptom of a cancer (see “Efficacy Measurement” below). Such effective amounts can be gauged in clinical trials as well as animal studies for a given agent.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of an anti-cancer agent calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

Combination Therapies: In some embodiments, an anti-cancer agent or drug selected using the methods and assays described herein is used in combination with the therapeutic use of at least one additional anti-cancer therapy, including at least one additional anti-cancer agent or chemotherapeutic, X-rays, gamma rays or other sources of radiation to destroy cancer stem cells and/or cancer cells. Indeed, in some embodiments, the methods described herein permit the identification of one or more agents that effectively kills CSCs, and one or more different agents that effectively kills NSCCCs. In such instances, it is specifically contemplated that a combination therapy would combine these agents that effectively kill each cancer cell population.

When two therapeutically effective anti-cancer treatments (e.g., two different anti-cancer agents selected using the methods described herein) are administered to a subject concurrently, the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the cancer therapeutics may be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the components of the combination cancer therapeutics are not administered in the same pharmaceutical composition, it is understood that they can be administered in any order to a subject in need thereof. For example, a first prophylactically and/or therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof. In various embodiments, the cancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, one or more anti-cancer agents (or non-pharmacological treatment(s)) are administered within the same office visit. In another embodiment, the combination cancer therapeutics are administered at 1 minute to 24 hours apart.

In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The agent and/or other therapeutic agents, procedures or modalities can be administered during periods of active disorder, or during a period of remission of acute cancer or less active disease. The agent can be administered before another treatment, concurrently with the treatment, post-treatment, or during remission of the disorder.

When administered in combination, the anti-cancer agent or drug selected using the assays described herein and at least one additional anti-cancer agent or drug selected using the assays described herein (e.g., second, third agent or a cocktail of 4 drugs or more), or all, can be administered in an amount or dose that is higher, lower or the same as the amount or dosage of each agent used individually, e.g., as a monotherapy. In certain embodiments, the administered amount or dosage of the anti-cancer agent, the at least one additional anti-cancer agent or drug, or all, is lower (e.g., at least 20%, at least 30%, at least 40%, or at least 50%) than the amount or dosage of each drug/agent used individually. In other embodiments, the amount or dosage of the anti-cancer agent or drug, the at least one additional anti-cancer agent or drug, or all, that results in a desired effect (e.g., reduction in tumor size, reduced growth rate, reduction in number of CSCs) is lower (e.g., at least 20%, at least 30%, at least 40%, or at least 50% lower) than the amount or dosage of each anti-cancer agent or drug individually required to achieve the same therapeutic effect.

In a specific embodiment, the combination therapies have the same mechanism of action. In another specific embodiment, the combination therapies each have a different mechanism of action. In one embodiment, the anti-cancer agent used in combination are from the same class of drug or from different classes.

### Pharmaceutical Compositions

An anti-cancer agent selected for a given subject using the methods and assays described herein can be administered as a pharmaceutical composition. Such pharmaceutical or therapeutic compositions can contain a physiologically tolerable carrier together with an active anti-cancer agent as described herein, dissolved or dispersed therein as an active ingredient. In a preferred embodiment, the pharmaceutical composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological or pharmaceutical composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition comprising at least one anti-cancer agent or drug can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

The use of drugs in a formulation already approved by the FDA are specifically contemplated herein for treatment of cancer in a subject.

### Efficacy Measurement

The efficacy of a given treatment for a cancer (including, but not limited to, leukemia) can be determined by the skilled clinician. However, a treatment is considered “effective treatment,” as the term is used herein, if any one or all of the signs or symptoms of the cancer is/are altered in a beneficial manner, or other clinically accepted symptoms or markers of disease are improved, or ameliorated, e.g., by at least 10% following treatment with an anti-cancer agent or combination thereof selected using the methods and assays described herein. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the cancer; or (2) relieving the disease, e.g., causing regression of symptoms; and (3) preventing or reducing the likelihood of the development of the disease, or preventing secondary diseases/disorders associated with the cancer (e.g., cancer metastasis).

An effective amount for the treatment of a disease means that amount which, when administered to a mammal in need thereof, is sufficient to result in effective treatment as that term is defined herein, for that disease. Efficacy of an agent can be determined by assessing physical indicators of the disease, such as e.g., pain, tumor size, tumor growth rate, blood cell count, etc.

The present invention may be as defined in any one of the following numbered paragraphs.

1. A high throughput functional cell assay comprising the steps of: (a) contacting aliquots of a biological sample from an individual having cancer each with individual members of a panel of therapeutic drugs or a combination thereof, the sample comprising a population of non-stem cell cancer cells and a population of cancer stem cells (CSCs); and (b) quantifying, respectively, cell viability of the population of CSCs and the population of non-stem cell cancer cells (NSCCCs).

2. A high throughput functional cell assay comprising the steps of: (a) contacting aliquots of a biological sample from an individual having cancer with individual members of a panel of anti-cancer agents, the sample comprising a population of non-stem cell cancer cells and a population of cancer stem cells (CSCs); and (b) quantifying, respectively, cell viability of the population of CSCs and the population of non-stem cell cancer cells (NSCCCs).

3. The assay of paragraph 1 or 2, wherein cell viability is assessed using a tetrazolium reduction assay, a resazurin reduction assay, a protease viability marker assay, a live cell protease assay, an ATP assay, a luciferase-based real-time assay, flow cytometry, or high content imaging.

4. The assay of paragraph 1, 2 or 3, further comprising a step, performed prior to steps (a) and (b), of seeding the aliquots of the biological sample in a plurality of wells.

5. The assay of any one of paragraphs 1-4, wherein the panel of anti-cancer agents comprises at least two drugs selected from a class or classes of drugs selected from the group consisting of: tyrosine kinase inhibitors, histone deacetylase inhibitors, cyclin-dependent kinase inhibitors, proteasome inhibitors, targeted inhibitor, PARP inhibitors, alkylating agents, cisplatinum compounds, anthracyclines, topoisomerase inhibitors, Flt-3 kinase inhibitors, and microtubule assembly inhibitors.

6. The assay of any one of paragraphs 1-5, wherein the panel of anti-cancer agents comprises at least 2 anti-cancer agents and no more than 1000 anti-cancer agents.

7. The assay of any one of paragraphs 1-6, wherein each individual member of the panel is tested with at least five different concentrations of the anti-cancer agent for each population.

8. The assay of paragraph 7, wherein a dose-response curve is generated for each individual member and each population using the data from the at least two different concentrations of the anti-cancer agent.

9. The assay of paragraph 8, wherein Area Under the Curve (AUC) is calculated for each individual member in the panel of anti-cancer agents and for each population from the respective dose-response curve for that anti-cancer agent.

10. The assay of any one of paragraphs 1-9, wherein the biological sample is obtained from the subject using a resection, biopsy, vacuum assisted biopsy, core needle biopsy or fine needle aspirate of a primary or metastatic tumor, or wherein the biological sample comprises a blood sample, a bone marrow aspiration, cerebrospinal fluid, pleural fluid, pericardial fluid, ascites fluid, urine, or isolated cells thereof.

11. The assay of any one of paragraphs 1-10, further comprising a step of ranking the individual members of the panel of anti-cancer agents based on their effect on cell viability for CSCs and/or NSCCCs.

12. The assay of any one of paragraphs 1-11, further comprising a step of comparing the cell viability for each anti-cancer agent on CSCs and/or NSCCCs to a reference.

13. The assay of any one of paragraphs 1-12, wherein quantifying step (b) comprises detecting signal from one or more markers permitting quantitative distinction between populations of CSCs and NSCCCs.

14. The assay of any one of paragraphs 1-13, further comprising a step of contacting the population of NSCCCs and the population of cancer stem cells (CSCs) with detectable probes that specifically bind and provide signal for the one or more markers.

15. The assay of paragraph 13 or 14, wherein the signal comprises fluorescent emission.

16. The assay of any one of paragraphs 1-15, wherein the markers permitting quantitative distinction between populations of CSCs and NSCCCs are selected from the markers in Table 1 or 2.

17. A method for selecting a personalized treatment for a subject having cancer, the method comprising: (a) performing a high throughput functional cell assay of any one of paragraphs 1-15 on a biological sample from a subject having cancer; and (b) on the basis of cell viability determined for CSCs and NSCCCs in (a), selecting a combination of at least two anti-cancer agent from the panel, the combination comprising a drug(s) effective to kill CSCs and a drug(s) effective to kill NSCCCs, thereby selecting a personalized treatment for the subject.

18. The method of paragraph 17, further comprising administering the combination of anti-cancer agents to the subject, thereby treating the subject's cancer.

19. The method of paragraph 17 or 18, wherein the cancer is refractory to or the subject has relapsed from prior treatment with a given anti-cancer agent.

20. The method of any one of paragraphs 17-19, wherein the CSCs comprise a leukemic stem cell, an acute myeloid leukemia stem cell, a brain cancer stem cell, a breast cancer stem cell, an ovarian cancer stem cell, a pancreatic cancer stem cell, a prostate cancer stem cell, a melanoma stem cell, a multiple myeloma stem cell, a colon cancer stem cell, an esophageal cancer stem cell, a stomach cancer stem cell, a lung cancer stem cell, a liver cancer stem cell, a head and neck squamous cell carcinoma stem cell, multiple myeloma stem cell, or a non-melanoma skin cancer stem cell.

21. A high throughput functional cell assay comprising the steps of: (a) isolating from a biological sample obtained from a subject having cancer, a population enriched for viable cancer stem cells (CSCs), (b) isolating from the same or a different biological sample obtained from the subject having cancer, a population enriched for viable non-stem cell cancer cells (NSCCCs); (c) contacting aliquots of the population enriched for CSCs with individual members of a panel of anti-cancer agents, (d) contacting aliquots of the population enriched for NSCCCs with individual members of a panel of anti-cancer agents, and (e) determining cell viability of the cells in each of the populations of step (c) and (d).

22. The assay of paragraph 21, wherein cell viability is assessed using a tetrazolium reduction assay, a resazurin reduction assay, a protease viability marker assay, a live cell protease assay, an ATP assay, a luciferase-based real-time assay, flow cytometry, or high content imaging.

23. The assay of paragraph 21 or 22, further comprising a step, performed prior to steps (c) and (d), of seeding CSCs in a first plurality of wells and a step of seeding NSCCCs in a second plurality of wells.

24. The assay of any one of paragraphs 20-23, wherein the panel of anti-cancer agents comprises at least two drugs selected from a class or classes of drugs selected from the group consisting of: tyrosine kinase inhibitors, histone deacetylase inhibitors, cyclin-dependent kinase inhibitors, proteasome inhibitors, targeted inhibitor, PARP inhibitors, alkylating agents, cisplatinum compounds, anthracyclines, topoisomerase inhibitors, Flt-3 kinase inhibitors, and microtubule assembly inhibitors.

25. The assay of any one of paragraphs 20-24, wherein the panel of anti-cancer agents comprises at least 2 anti-cancer agents and no more than 1000 anti-cancer agents.

26. The assay of any one of paragraphs 20-25, wherein each individual member of the panel is tested in at least five different concentrations of the anti-cancer agent for each population.

27. The assay of any one of paragraphs 20-26, wherein a dose-response curve is generated for each individual member and each population using the data from the at least five different concentrations of the anti-cancer agent.

28. The assay of any one of paragraphs 20-27, wherein Area Under the Curve (AUC) is calculated for each individual member in the panel of anti-cancer agents and for each population from the respective dose-response curve for that anti-cancer agent.

29. The assay of any one of paragraphs 20-28, wherein the biological sample is obtained from the subject using a resection, biopsy, vacuum assisted biopsy, core needle biopsy or fine needle aspirate of a primary or metastatic tumor or wherein the biological sample comprises a blood sample, a bone marrow aspiration, cerebrospinal fluid, pleural fluid, pericardial fluid, ascites fluid, urine, or isolated cells thereof.

30. The assay of any one of paragraphs 20-29, further comprising a step of ranking the individual members of the panel of anti-cancer agent based on their effect on cell viability for each population.

31. The assay of any one of paragraphs 20-30, further comprising a step of comparing the cell viability for each anti-cancer agent and for each population to a reference.

32. A method for selecting a personalized treatment for a subject having cancer, the method comprising: (a) isolating from a biological sample obtained from a subject having cancer, a population enriched for cancer stem cells (CSCs), (b) isolating from the same or a different biological sample obtained from the subject having cancer, a population enriched for non-stem cell cancer cells (NSCCCs); (c) contacting aliquots of the population enriched for CSCs with individual members of a panel of anti-cancer agents, (d) contacting aliquots of the population enriched for cancer cells with individual members of a panel of anti-cancer agents or combinations thereof, (e) determining cell viability of the cells in each of the populations of step (c) and (d), (f) selecting, based on criteria comprising reduced cell viability, at least one anti-cancer agent, thereby selecting a personalized treatment for the subject having cancer.

33. The method of paragraph 32, further comprising administering the at least one anti-cancer agent selected in step (f) to the subject having cancer.

34. The method of paragraph 32 or 33, further comprising a step, performed prior to steps (c) and (d), of seeding CSCs in a plurality of wells and a step of seeding cancer cells in a plurality of wells.

35. The method of any one of paragraphs 31-34, wherein the panel of anti-cancer agents comprises at least two drugs selected from a class or classes of drugs selected from the group consisting of: tyrosine kinase inhibitors, histone deacetylase inhibitors, cyclin-dependent kinase inhibitors, proteasome inhibitors, targeted inhibitor, PARP inhibitors, alkylating agents, cisplatinum compounds, anthracyclines, topoisomerase inhibitors, Flt-3 kinase inhibitors, and microtubule assembly inhibitors.

36. The method of any one of paragraphs 31-35, wherein the panel of anti-cancer agents comprises at least 2 anti-cancer agents and no more than 1000 anti-cancer agents.

37. The method of any one of paragraphs 31-36, further comprising a step of obtaining the biological sample from the subject having cancer.

38. The method of any one of paragraphs 31-37, wherein the cancer is refractory to or the subject has relapsed from prior treatment with a given anti-cancer agent.

39. The method of any one of paragraphs 31-38, wherein the CSCs comprise a leukemic stem cell, an acute myeloid leukemia stem cell, a brain cancer stem cell, a breast cancer stem cell, an ovarian cancer stem cell, a pancreatic cancer stem cell, a prostate cancer stem cell, a melanoma stem cell, a multiple myeloma stem cell, a colon cancer stem cell, an esophageal cancer stem cell, a stomach cancer stem cell, a lung cancer stem cell, a liver cancer stem cell, a head and neck squamous cell carcinoma stem cell, multiple myeloma stem cell, or a non-melanoma skin cancer stem cell.

40. The method of any one of paragraphs 31-39, wherein the biological sample lacks red blood cells.

41. The method of any one of paragraphs 31-40, further comprising a step of removing red blood cells.

42. The method of any one of paragraphs 31-41, further comprising a step of ranking the individual members of the panel of anti-cancer agents based on their effect on cell viability for each population.

43. The method of any one of paragraphs 31-42, further comprising a step of comparing the cell viability for each anti-cancer agent and for each population to an appropriate reference.

44. The method of any one of paragraphs 31-43, wherein each individual member of the panel is tested with at least two different concentrations of the anti-cancer agent in each population.

45. The method of any one of paragraphs 31-44, wherein a dose-response curve is generated for each individual member and each population using the data from the at least five different concentrations of the anti-cancer agent.

46. The method of any one of paragraphs 31-45, wherein Area Under the Curve (AUC) is calculated for each individual member in the panel of anti-cancer agents and for each population from the respective dose-response curve for that anti-cancer agent.

47. The method of any one of paragraphs 31-46, wherein the biological sample is obtained from the subject using a resection, biopsy, vacuum assisted biopsy, core needle biopsy or fine needle aspirate of a primary or metastatic tumor or wherein the biological sample comprises a blood sample, a bone marrow aspiration, cerebrospinal fluid, pleural fluid, pericardial fluid, ascites fluid, urine, or isolated cells thereof.

48. The method of any one of paragraphs 31-47, wherein step (f) is performed by a skilled clinician.

49. The method of any one of paragraphs 31-48, wherein the results of step (f) are communicated to a skilled clinician.

50. The method of any one of paragraphs 31-49, wherein the population enriched for non-stem cell cancer cells comprises less than 20% CSCs and/or wherein the population enriched for CSCs comprises less than 20% NSCCs.

51. The method of any one of paragraphs 31-50, wherein the steps (a)-(f) are repeated at least once.

52. The method of any one of paragraphs 31-51, wherein the steps (a)-(g) are repeated in the presence of a different panel of anti-cancer agents.

53. A method for monitoring treatment efficacy in a subject being treated for cancer, the method comprising: (a) isolating from a biological sample obtained from a subject being treated for cancer with an anti-cancer agent, a population enriched for cancer stem cells (CSCs), (b) isolating from the same or a different biological sample obtained from the subject being treated for cancer with an anti-cancer agent(s), a population enriched for cancer cells; (c) contacting aliquots of the population enriched for CSCs with the anti-cancer agent(s), (d) contacting aliquots of the population enriched for non-stem cell cancer cells with the anti-cancer agent(s), and (e) determining cell viability of the cells in each of the populations of step (c) and (d), wherein a reduction in cell viability in the presence of the anti-cancer agent as compared to an untreated or vehicle treated aliquot of the same cell population indicates that the anti-cancer agent is efficacious in the subject being treated for cancer.

54. The method of paragraph 53, wherein the method is repeated at least once while the subject is being treated for cancer.

55. The method of paragraph 53 or 54, wherein the method is repeated weekly, monthly, every 6 months, or annually.

56. The method of any one of paragraphs 53-55, further comprising the steps of: (a) contacting aliquots of the population enriched for CSCs with individual members of a panel of anti-cancer agents, (b) contacting aliquots of the population enriched for cancer cells with individual members of a panel of anti-cancer agents, (c) determining cell viability of the cells in each of the populations of step (a) and (b), (d) selecting, based on criteria comprising reduced cell viability assessed in step (c), the same or a different anti-cancer agent or combination thereof than the anti-cancer agent(s) used to treat cancer in the subject.

57. A method for treating a subject having acute myeloid leukemia (AML), the method comprising: (a) isolating from a biological sample comprising white blood cells obtained from a subject having AML, a population enriched for leukemic stem cells (LSCs), (b) isolating from the same or a different biological sample comprising white blood cells obtained from the subject having AML, a population enriched for blast cells; (c) contacting aliquots of the population enriched for LSCs with individual members of a panel of anti-cancer agents or a combination thereof to determine the susceptibility of the LSCs to each drug or combination of drugs, (d) contacting aliquots of the population enriched for blast cells with individual members of a panel of anti-cancer agents or a combination thereof to determine the susceptibility of the blast cells to each drug or combination thereof, (e) selecting at least one drug from step (c) and/or step (d) to which the LSCs and/or blast cells are determined to be susceptible, and (0 administering the at least one drug selected in step (e) to the subject having AML, thereby treating the subject having AML.

58. The method of paragraph 57, further comprising a step, performed prior to steps (c) and (d), of seeding LSCs in a plurality of wells and a step of seeding blast cells in a plurality of wells.

59. The method of paragraph 57 or 58, wherein the panel of anti-cancer agents comprises at least two drugs selected from a class or classes of drugs selected from the group consisting of: tyrosine kinase inhibitors, histone deacetylase inhibitors, cyclin-dependent kinase inhibitors, proteasome inhibitors, targeted inhibitor, PARP inhibitors, alkylating agents, cisplatinum compounds, anthracyclines, topoisomerase inhibitors, Flt-3 kinase inhibitors, and microtubule assembly inhibitors.

60. The method of any one of paragraphs 57-59, wherein the panel of anti-cancer agents comprises at least 2 anti-cancer agents and no more than 1000 anti-cancer agents.

61. The method of any one of paragraphs 57-60, further comprising a step of obtaining the biological sample comprising white blood cells from a subject having AML.

62. The method of any one of paragraphs 57-61, wherein the subject having AML is refractory to or has relapsed from conventional AML treatment.

63. The method of any one of paragraphs 57-62, wherein the conventional AML treatment comprises treatment with cytarabine and an anthracycline.

64. The method of any one of paragraphs 57-63, wherein the biological sample comprising white blood cells is a CD34+ enriched blast cell population.

65. The method of any one of paragraphs 57-64, wherein at least 75% of the cells in the CD34+ enriched blast cell population are CD34+ blast cells.

66. The method of any one of paragraphs 57-65, wherein the biological sample lacks red blood cells.

67. The method of any one of paragraphs 57-66, further comprising a step of removing red blood cells.

68. The method of any one of paragraphs 57-67, wherein each anti-cancer agent in the panel of anti-cancer agents is tested for each population using at least two different concentrations of the anti-cancer agent.

69. The method of any one of paragraphs 57-68, wherein each anti-cancer agent in the panel of anti-cancer agents is tested for each population using at least five different concentrations of the anti-cancer agent.

70. The method of any one of paragraphs 57-69, wherein a dose-response curve is generated for each population using the data from the at least five different concentrations of the anti-cancer agent.

71. The method of any one of paragraphs 57-70, wherein Area Under the Curve (AUC) is calculated for each anti-cancer agent in the panel of drugs for each population from the respective dose-response curve for that anti-cancer agent.

72. The method of any one of paragraphs 57-71, further comprising comparing the AUC for each anti-cancer agent in the panel of drugs for each population to an AUC calculated from the dose-response curve of mitomycin C for the same population.

73. The method of any one of paragraphs 57-72, wherein (i) an AUC^{stem mean}>AUC^{blast mean} for an anti-cancer agent in the panel indicates that the blast cells are susceptible to the anti-cancer agent, or (i) an AUC^{stem mean}<AUC^{blast mean} for an anti-cancer agent in the panel indicates that the LSC cells are susceptible to the anti-cancer agent.

74. The method of any one of paragraphs 57-73, wherein step (e) is performed by a skilled clinician.

75. The method of any one of paragraphs 57-74, wherein the results of step (e) are communicated to a skilled clinician.

76. The method of any one of paragraphs 57-75, wherein the population enriched for LSCs comprises at least 75% LSC cells comprising a marker profile of CD34⁺ CD38^{lo/-} CD123⁺.

77. The method of any one of paragraphs 57-76, wherein the population enriched for blast cells comprises less than 20% LSCs and/or the population enriched for LSCs comprises less than 20% blast cells.

78. The method of any one of paragraphs 57-78, wherein the biological sample comprising white blood cells is fractionated by FACS.

79. The method of any one of paragraphs 57-78, further comprising a step of fractionating the biological sample comprising white blood cells for viable cells by the characteristics of CD45^{dim}, side scatter^{lo}.

80. The method of any one of paragraphs 57-79, wherein the method is repeated at least once.

81. The method of any one of paragraphs 57-80, wherein the method is repeated at least once using a different panel of anti-cancer agent.

### Examples

### SUMMARY

Despite established clonal heterogeneity, with few exceptions, the same two-drug induction regimen (cytarabine+an anthracycline) has remained standard of care for 40 years. Poor long term survival, toxicity of typical multi-agent chemotherapy regimens, and molecular and cytogenetic heterogeneity have led to the development of precision medicine approaches to the treatment of AML. Early studies that utilize drug susceptibility by high throughput screening (HTS) have been reported. While initial data have proven this work is feasible and have suggested correlations with treatment response, this approach has not yet incorporated drug sensitivity data of the LSC fraction. To date, an ex vivo HTS against murine model T-ALL pre leukemic stem cells was shown to be feasible, but the rarity of this fraction in the leukemic patient limited screening (Gerby B et al. (2016) J Clin Invest 126:4569-4584). In this study, a HTS approach is extended to prove that it is feasible to gather LSC viability data using patient samples and to illustrate the differences in vitro drug susceptibility for blasts vs. LSCs in individual patients. It is hypothesized that durable response is more tightly correlated with different therapies.

In an initial study, the drug susceptibility of LSCs isolated by fluorescence activated cell sorting (FACS) was compared to that of AML leukemic blasts from which they were derived in a high throughput screen to ensure that drug choices made on the basis of functional screening would eradicate LSCs. One AML patient sample engrafted in NODscidIL2Rgc−/− mice was also used to compare a highly proliferative engrafted subclone to the original AML blasts. It was found that AML blasts and LSCs exhibited divergent drug susceptibility patterns. Seven of 11 drugs conventionally used in the treatment of AML were blast-specific (n=5) or trended toward blast-specific (n=2), whereas LSCs were resistant to all but one of these drugs, indicating a possible mechanism for post-treatment relapse or primary refractoriness. The engrafted subclone was also nearly uniformly resistant to all drugs tested and possessed three new deleterious mutations, indicating a genetic basis for resistance. Of note, 12 drugs were identified from 8 classes defined by mechanism of action that may preferentially target LSCs as compared to blasts. Incorporation of the results of functional drug screening focused on LSCs into individualized treatment for AML may identify patient-specific therapeutic approaches that improve outcomes.

### Materials and Methods

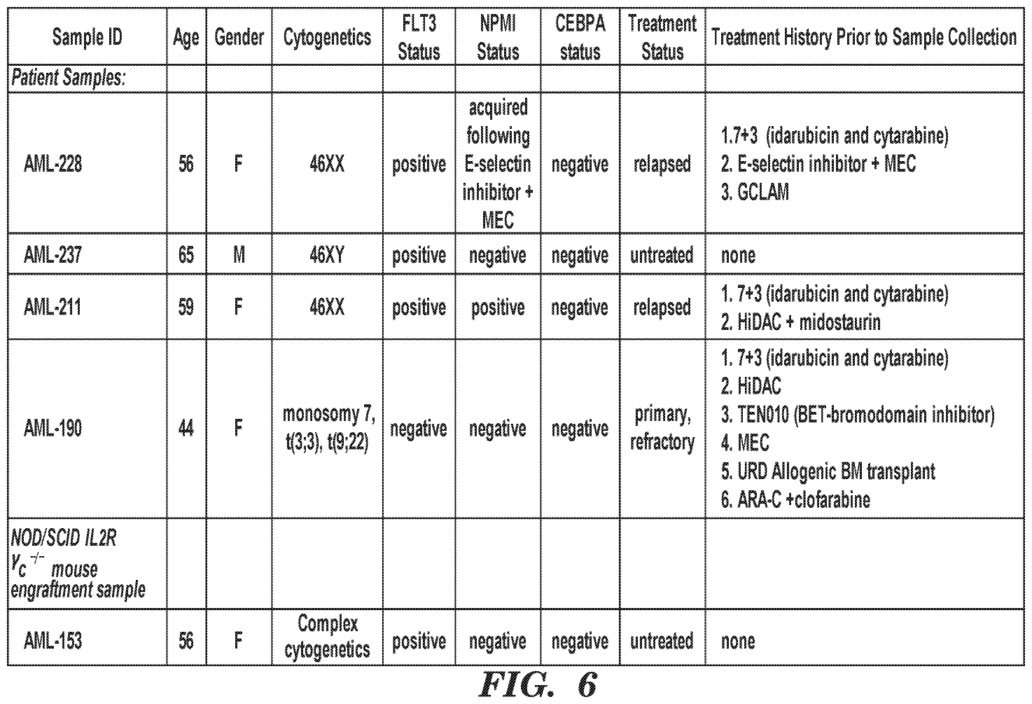

Patient samples: Clinical characteristics for 6 AML patient samples (2 fresh, 4 cryopreserved peripheral blood) are summarized in FIG. 6. Five of these samples (patient samples) were subjected to cell separations and screened directly while the sixth sample (NOD/SCID IL2R γc−/− mouse engraftment sample) was tested after isolation of human cells from a xenograft model. Three patients had newly diagnosed AML with FLT3 ITD mutations and two of the patients had a complex karyotype. Two patients had relapsed AML with a FLT3 ITD mutation, with most recent complete remission (CR) duration of <6 months, and one patient had primary refractory AML with complex karyotype including monosomy 7, t(3;3), and t(9;22), had failed 4 induction attempts and had relapsed after hematopoietic cell transplant. Xenograft studies were performed for one of the patients with de novo AML with FLT3 ITD and complex karyotype.

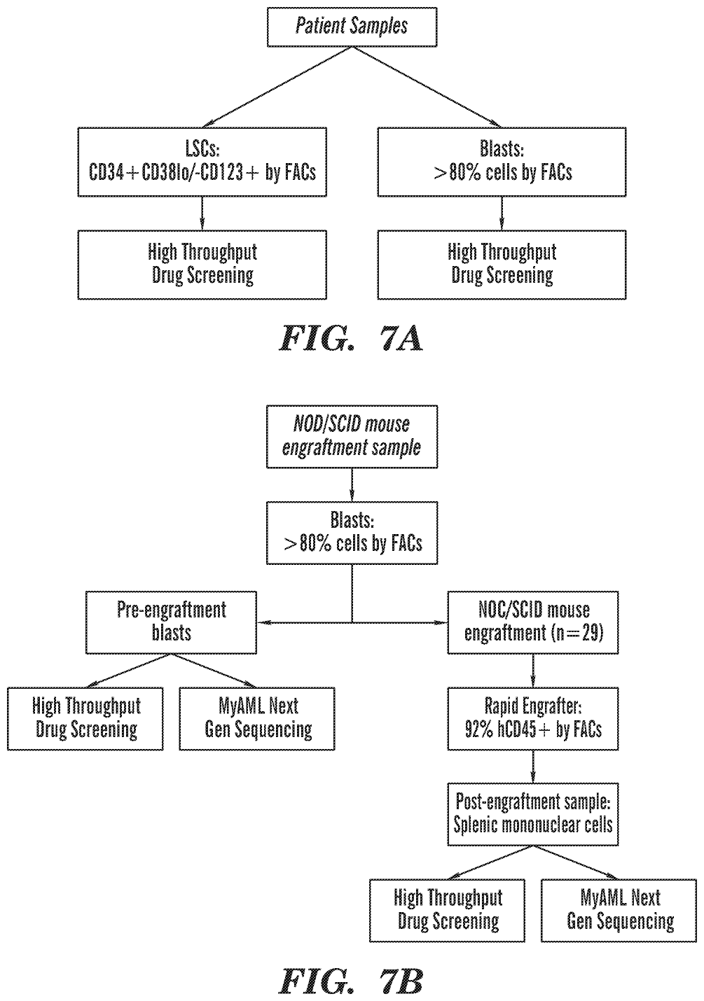

A schematic of experiments by sample is described in FIG. 7A.

Isolation of blasts and FACS isolation of LSCs: CD34+ blasts were enriched to >90% using immunomagnetic beads (QuadroMACS). Fluorescence activated cell sorting was performed in a biosafety cabinet enclosed FACS-Aria II (Becton-Dickinson; San Jose, Calif.) housed in the University of Washington Core Cell Analysis Facility where voltage was standardized and calibration was performed daily. Mononuclear cells were stained with allophycocyanin H7 (APC-H7) conjugated anti-human CD45, allophycocyanin (APC) conjugated anti-human CD34, phycoerythrin-Cy7 (PE-Cy7) conjugated anti-human CD38 and phycoerythrin (PE) conjugated anti-human CD123 (all from BD Biosciences Pharmingen, San Jose, Calif.). Initially a blast gate was applied for forward scatter vs. side scatter, then sequential gates for CD45 vs. side scatter, CD34+ and CD38^{low or neg}, then CD123+ cells to yield the LSCs. The CD38low population was defined by CD38 above the isotype control and below that of mature precursors. Flow cytometry data analysis was performed using FlowJo software (Tree Star, Inc. Ashland, Oreg.).

Murine xenografts: NODscid IL2Rgc−/− (NOD.Cg-Prkdcscid Il2rgtm1Wjl/SzJ) mice were obtained from Jackson Laboratories (Bar Harbor, Me.). They were maintained and bred in a specific pathogen free facility at the University of Washington, Seattle, Wash. Animal care and study protocols were approved by the Institutional Animal Care and Use Committee (IACUC) of University of Washington School of Medicine.

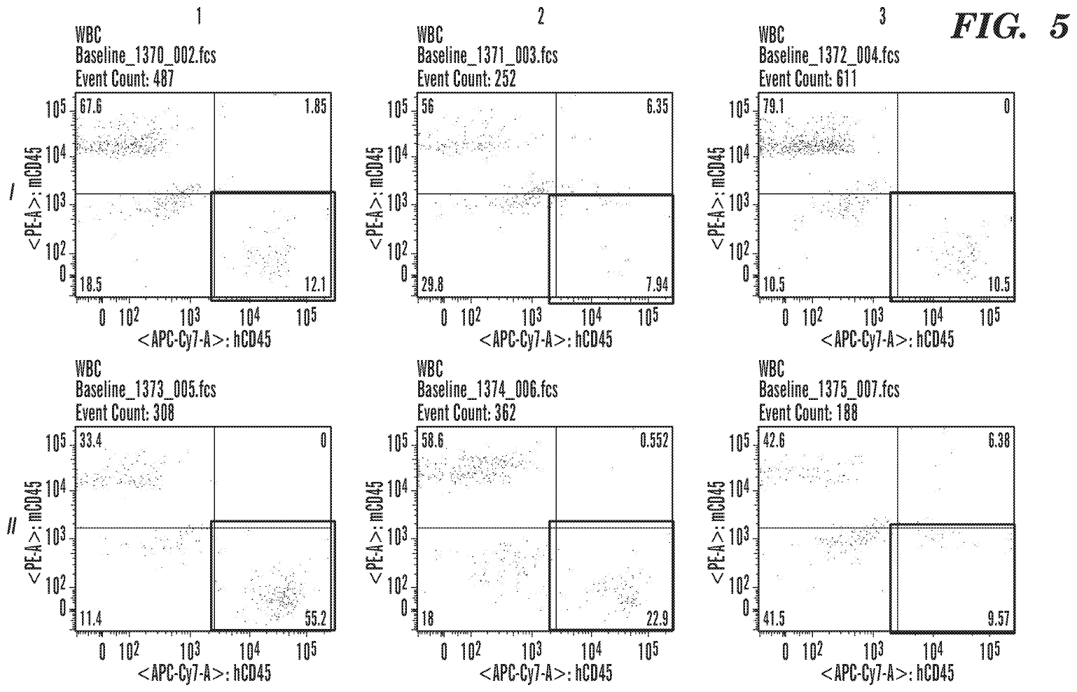

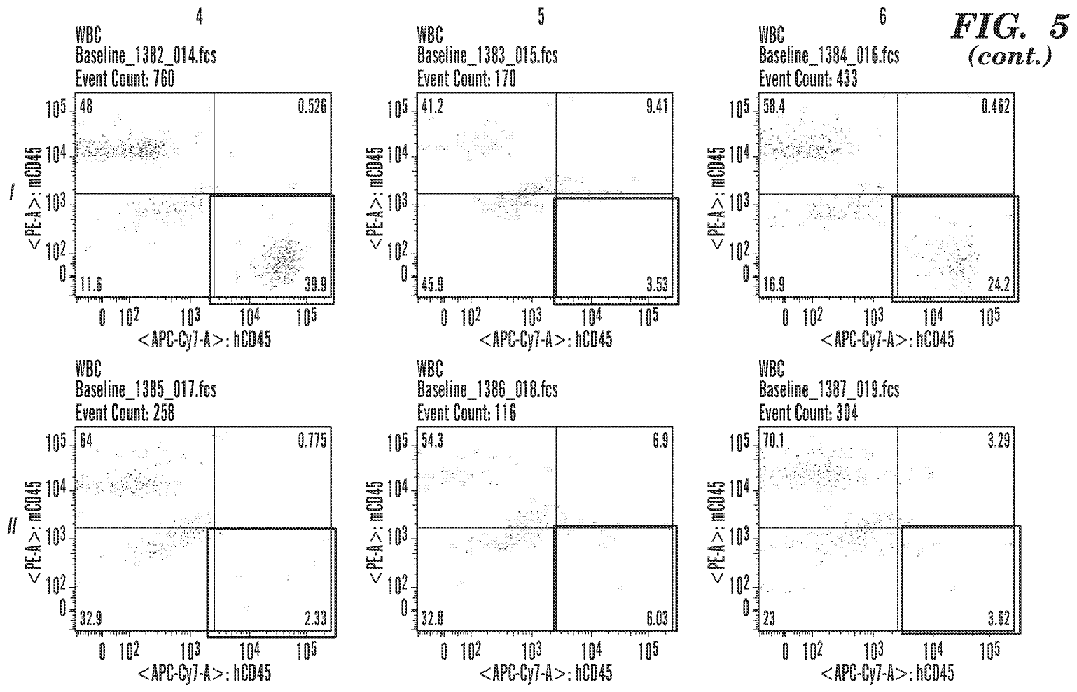

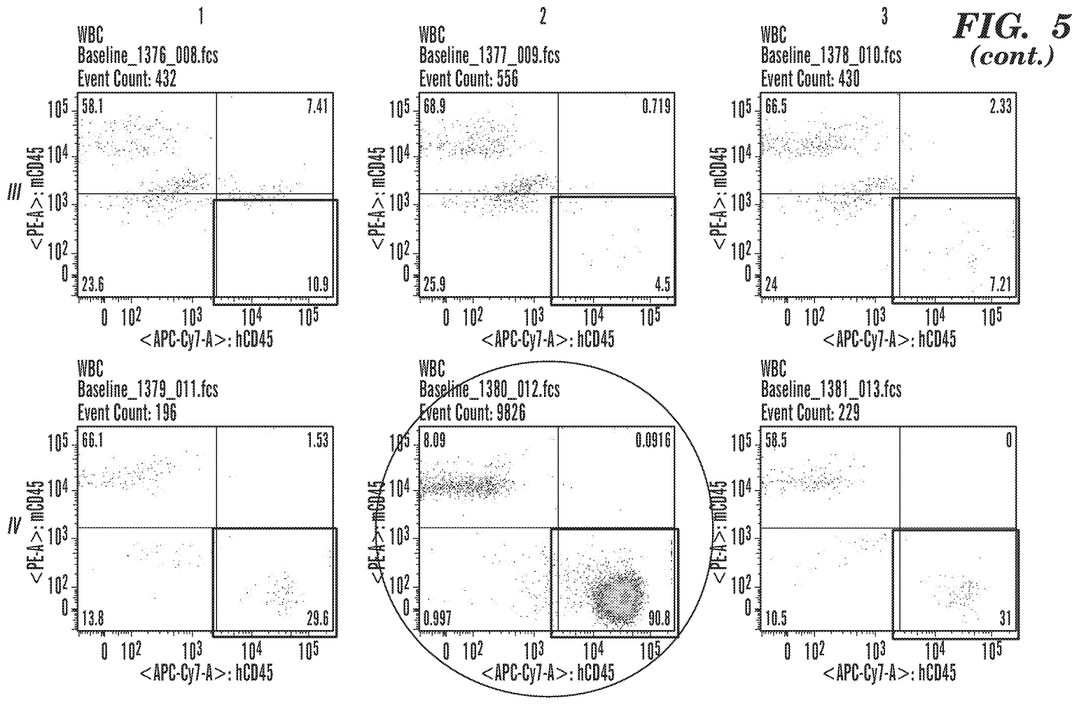

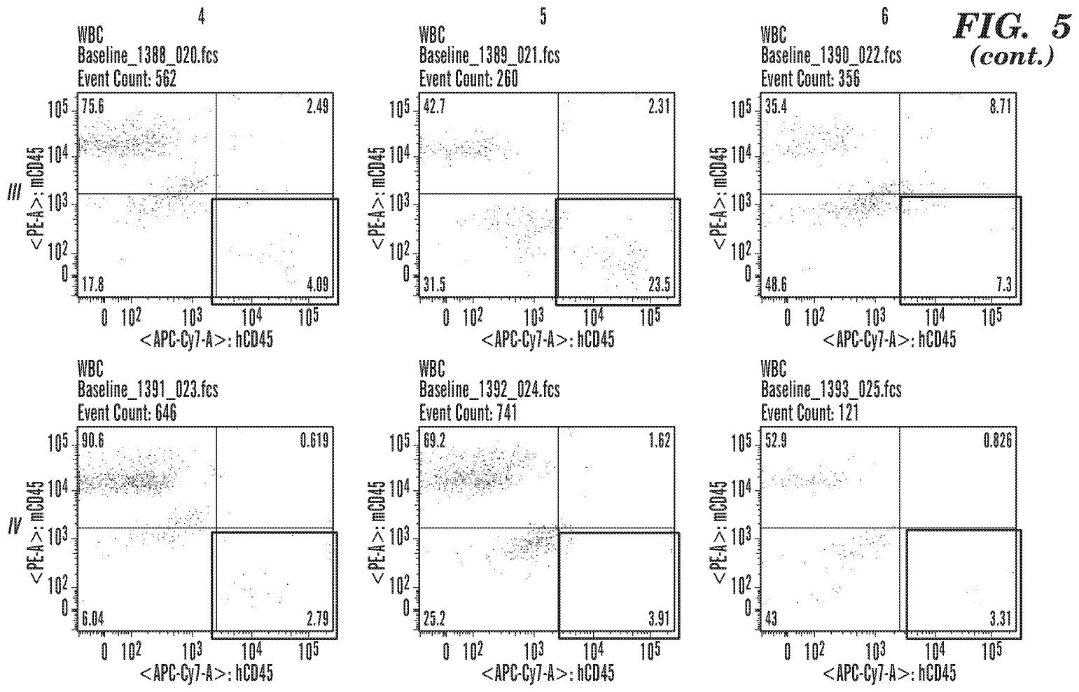

Twenty-nine Nod-Scid mice at 6-8 weeks of age and of both genders were administered a sub lethal dose of 300 cGy total body irradiation, then received 10 million mononuclear cells from a single AML patient IV by tail vein injection. The antibiotic, Baytril®, was added to the water for two weeks after irradiation. Analysis of engraftment was conducted by flow cytometry of blood samples. The cells were labeled with antibodies to mouse and human CD45, and isotype control antibodies (BD Biosciences Pharmingen, San Jose, Calif.). At three weeks, the human proportion of cells from the sample from one male mouse far exceeded the others, 91% hCD45 positive cells; all other mice had less robust engraftment (FIG. 5). The degree of engraftment and out growth was a surrogate for leukemia initiation and propagation potential; therefore, splenic mononuclear cells from this mouse engrafted with human AML were collected for functional drug screen and mutation analysis.

Functional Drug Screen: A CLIA-validated in vitro sensitivity assay was performed that included both FDA approved and investigational drugs. ‘Oncopane11’ comprised of 153 drugs was used for HTS of patient samples and an earlier generation panel of 160 drugs was used for HTS of the NOD/SCID IL2R γc−/− mouse engraftment sample. As detailed above, for the 4 patient samples, using FACS, the blast population (CD45^{dim}, side scatter^{low}) was confirmed to be >90% of the viable white blood cell fraction, and the LSC subpopulation (CD34⁺CD38^{low or neg}CD123⁺) was isolated. For the NOD/SCID IL2R γc−/− mouse engraftment sample, both the pre-engraftment AML blasts (>90% blasts by FACs) and splenic mononuclear cells (91% hCD45+ engrafting subclone as above) were assayed. After overnight incubation on matrix protein coated 384 well plates, drugs were added to each well using a CyBio Well Vario liquid handler (Amalytik Jena). The cell populations were analyzed for survival after a 72-hour exposure to 8 customized drug concentrations (within the range of 5 pM to 100 microM) of each drug spanning 4 logs. Post exposure viability was measured using CellTiter Glo luminescent reagent (Promega, Madison, Wis.) per manufacturer protocol, then the plates were analyzed with the Envision Multi-label plate reader (Perkin Elmer, Waltham, Mass.). XLFit software (IDBS, Guildford, Surrey, United Kingdom) was used to analyze the data and generate dose response curves based on standard 4-parameter logistic fit. For each plate, data were normalized to DMSO 100% viability and blank controls.

Area under the curve (AUC) was calculated for each dose response curve. For the 4 patient samples, two-sample two-tailed t-tests were used for group comparisons of AUC values to determine drug specificity and efficacy.

Drug specificity was defined by group comparisons between AUCs for each drug and cell type across patient samples. For blast specific drugs AUCstem mean>AUCblast mean and for LSC specific drugs AUC^{stem mean}<AUC^{blast} mean (p<0.1). Drugs were determined to be effective vs. LSCs if they had an AUC^{stem} mean less than the mitomycin C AUC^{blast} mean (p<0.1); similarly, drugs were determined to be effective vs. blasts if they had an AUC^{blast} mean less than the mitomycin C AUC^{blast} mean (p<0.1). This approach was selected to detect differences within the small sample size, while still maintaining statistical stringency and capturing trends observed in the data set. Group comparisons for categorical variable were completed using Fisher's exact test.

Mutation analysis: DNA isolated with an Illumina kit from patient-derived enriched blast samples and leukemia stem cell (LSC) fractions isolated by FACS were subjected to mutation analysis by the MyAML™ CLIA validated next generation sequencing assay, which screens for mutations in 194 genes associated with AML.

For the NOD/SCID IL2R γc−/− engrafted sample, the pre-engraftment blasts and post engraftment subclone were analyzed. The variant allele fractions were provided, as well as the predicted impact of observed mutations on protein function as determined by SIFT and PolyPhen2 tools. All mutations with variant allele fractions>2.5% were compared for the blast and LSC fractions from the same patients. Mutations unique to each of the populations were identified, as well as those in common to the two populations.

### Results

### High Throughput Drug Screen

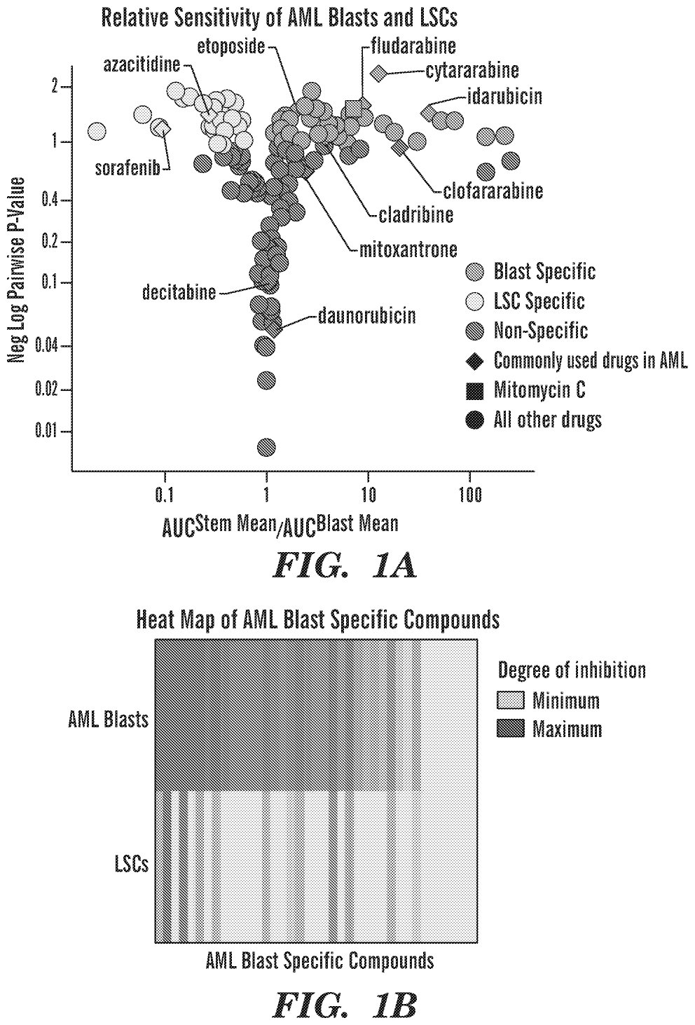

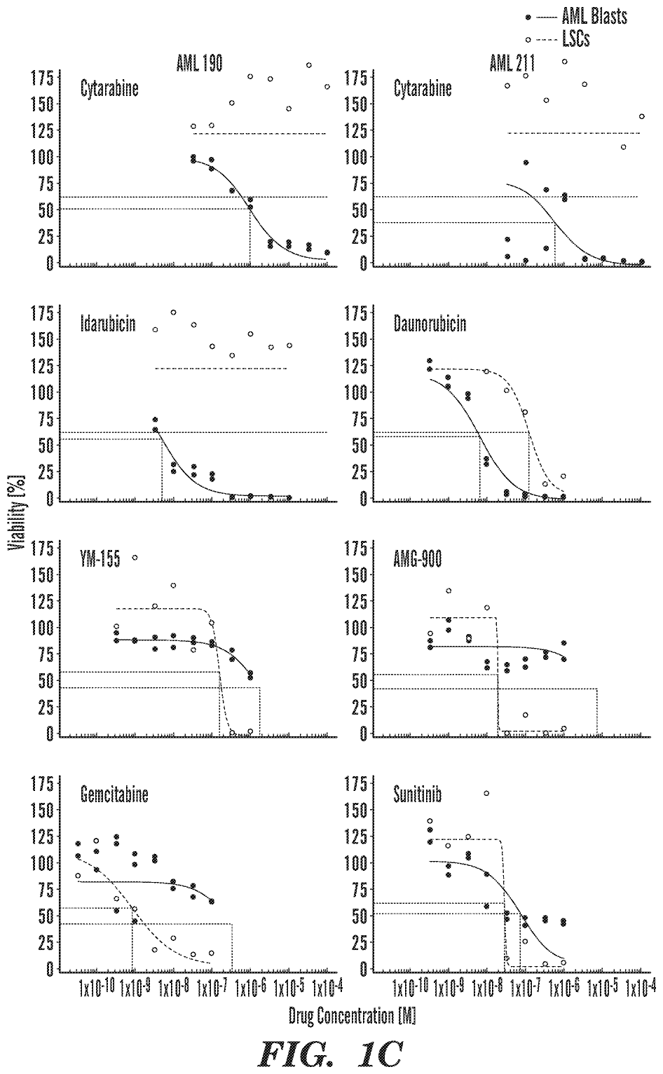

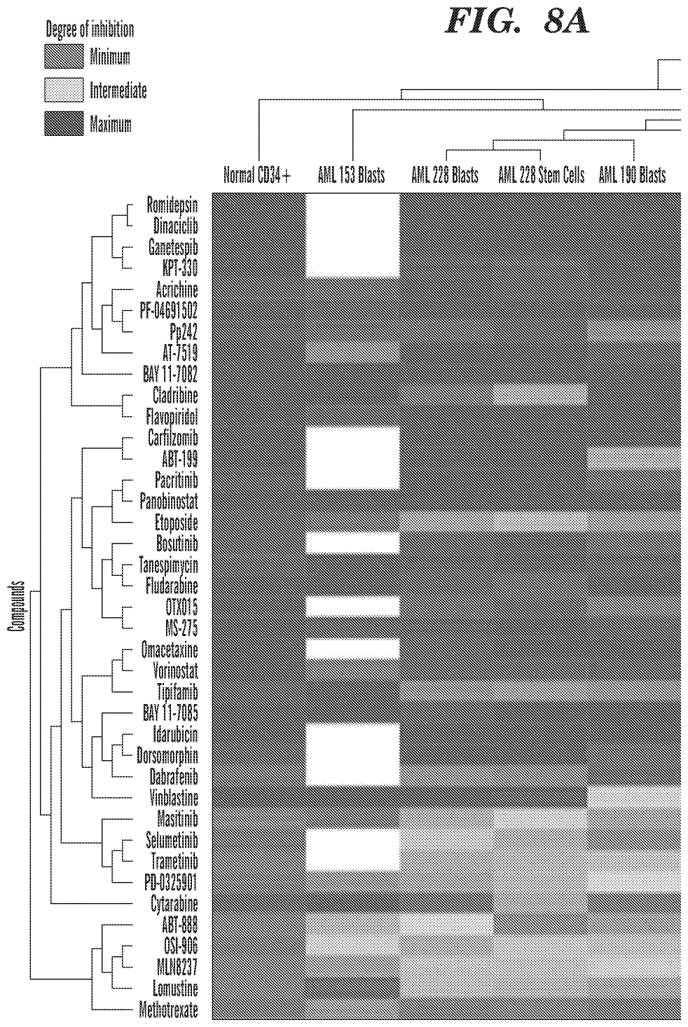

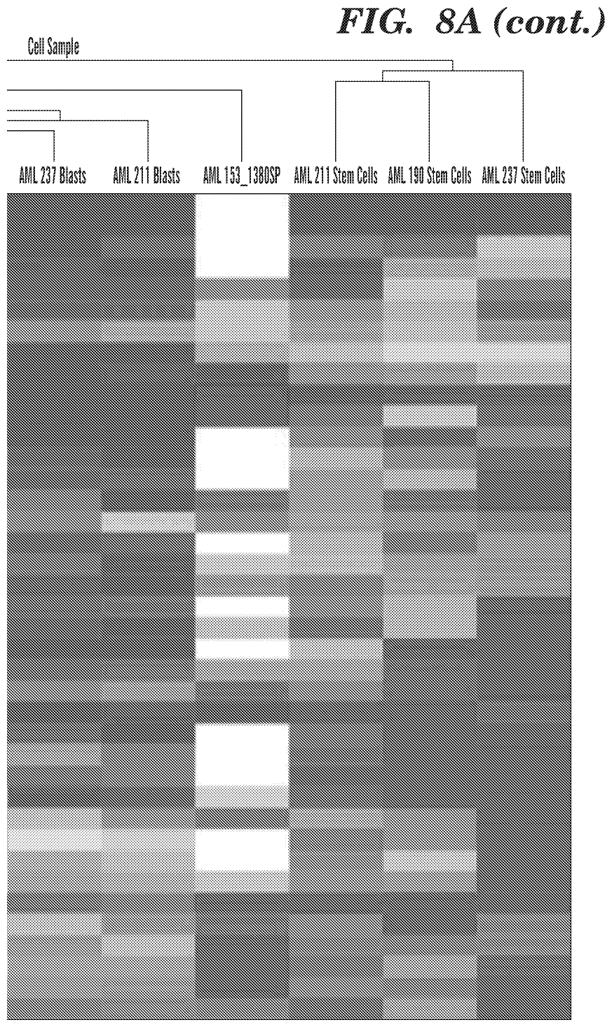

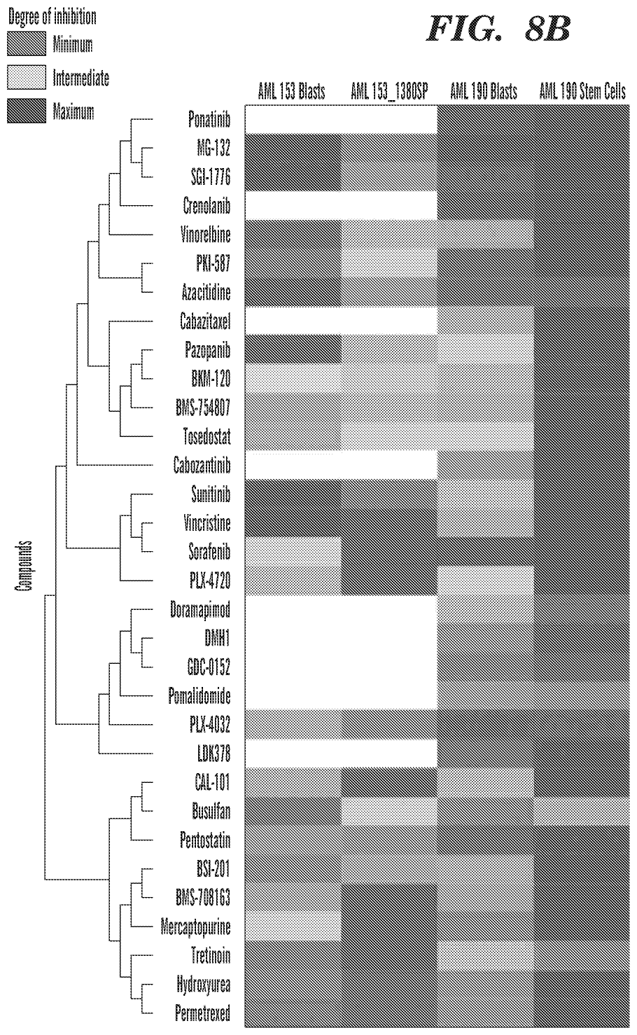

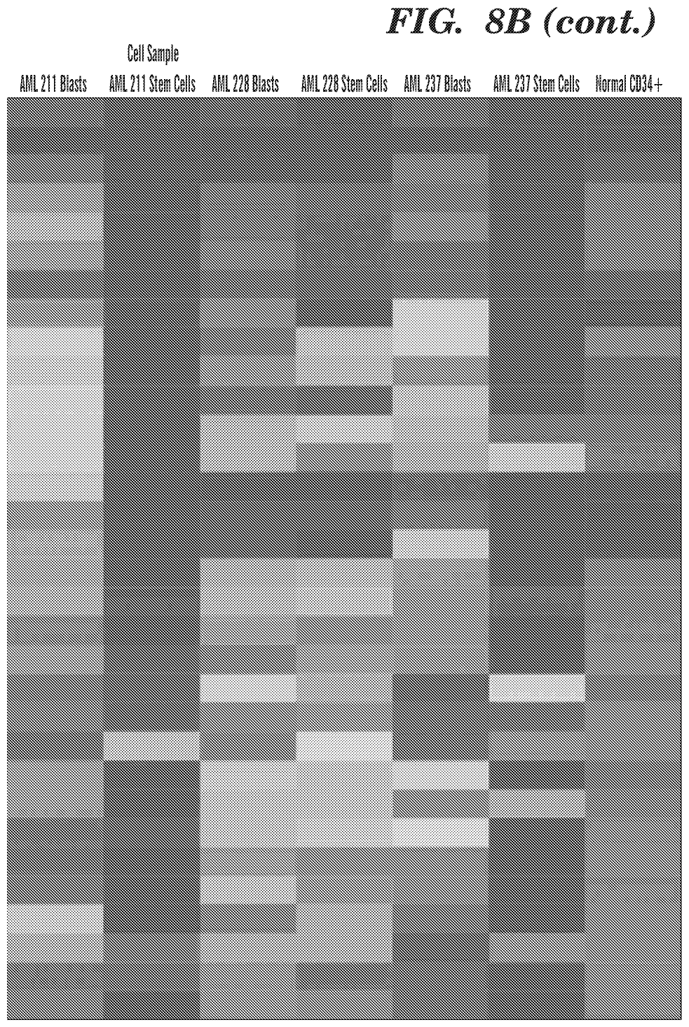

Blasts vs. FACS sorted LSCs: For the patient samples, the proportion of sorted LSCs (CD34+CD38lo/−CD123+) within the bulk blast cell population ranged from 7.0% to 25.6% (mean 14.7%). Aggregate viability data using the patient samples identified 40 drugs as blast cell specific (AUCstem mean>AUCblast mean, pairwise p-value<0.1, i.e. blast cells were more susceptible than stem cells) and 32 drugs as leukemia stem cell specific (AUCstem mean<AUCblast mean, pairwise p-value<0.1). 11 drugs commonly used in AML today were included in the specificity analysis; of these, 5 (cytarabine, cladribine, idarubicin, etoposide, fludarabine) were blast cell specific (pairwise p-value<0.1), 2 (clofarabine and mitoxantrone) trended towards blast cell specific (pairwise p-values: 0.11 and 0.21 respectively), 2 (daunorubicin and decitabine) were non-specific, and 2 (sorafenib and azacitidine) were LSC specific (pairwise p-value<0.1). Also, mitomycin C, a non-standard of care drug of interest, was blast cell specific (pairwise p-value<0.05) (FIG. 1). Compared to blasts and LSCs, normal CD34+ cells were more susceptible to standard chemotherapy drugs (FIGS. 8A-8B).

Aggregate viability data using the patient samples were also used to assess drug effect. LSC specificity (AUCstem mean<AUCblast mean, pairwise p-value<0.1) did not necessarily translate into in vitro efficacy against LSCs (AUCstem mean<mitomycin C's AUCblast mean, pairwise p-value<0.1); for example, azacitidine, while LSC specific, did not result in appreciable cytotoxicity in vitro, likely reflecting that its mechanism of action is not observed in a 72 h timeframe. LSCs were effectively killed by 12 drugs from 8 drug classes including only one drug used in AML (off-label), sorafenib (Table 3).

| Blast Cell Specific | Stem Cell Specific |
|---|---|
| Compounds | Compounds |
| ABT-199 | Azacitidine |
| ABT-888 | BKM-120 |
| Acrichine | BMS-708163 |
| AT-7519 | BMS-754807 |
| BAY 11-7082 | BSI-201 |
| BAY 11-7085 | Busulfan |
| Bosutinib | Cabazitaxel |
| Carfilzomib | Cabozantinib |
| Cladribine | CAL-101 |
| Cytarabine | Crenolanib |
| Dabrafenib | DMH1 |
| Dinaciclib | Doramapimod |
| Dorsomorphin | GDC-0152 |
| Etoposide | Hydroxyurea |
| Flavopiridol | LDK378 |
| Fludarabine | Mercaptopurine |
| Ganetespib | MG-132 |
| Idarubicin | Pazopanib |
| KPT-330 | Pemetrexed |
| Lomustine | Pentostatin |
| Masitinib | PKI-587 |
| Methotrexate | PLX-4032 |
| Mitomycin C | PLX-4720 |
| MLN8237 | Pomalidomide |
| MS-275 | Ponatinib |
| Omacetaxine | SGI-1776 |
| OSI-906 | Sorafenib |
| OTX015 | Sunitinib |
| Pacritinib | Tosedostat |
| Panobinostat | Tretinoin |
| PD-0325901 | Vincristine |
| PF-04691502 | Vinorelbine tartrate |
| Pp242 | |
| Romidepsin | |
| Selumetinib | |
| Tanespimycin | |
| Tipifarnib | |
| Trametinib | |
| Vinblastine | |
| Vorinostat | |

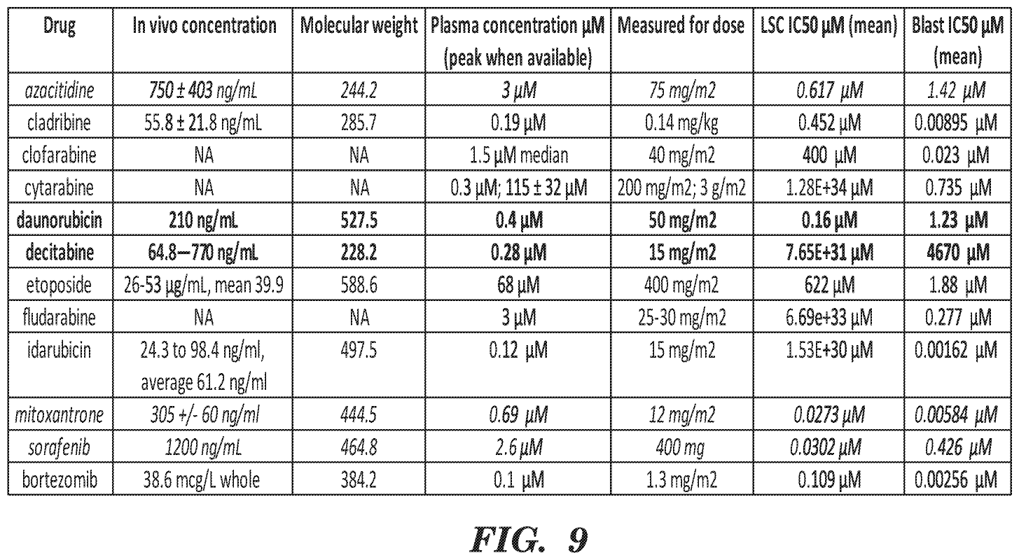

Of the 11 drugs commonly used in AML, 8 were typical chemotherapy drugs. Five of these compounds were effective against blasts (AUCblast mean<mitomycin C's AUCblast mean, pairwise p-value<0.1), but none were effective against LSCs (p-value: 0.0256). Further, for the 8 chemotherapy agents, the effective drug concentration in vitro (mean IC50) was compared to expected plasma concentration in patients. For six of these drugs (cladribine, cytarabine, clofarabine, etoposide, fludarabine and idarubicin), it was observed that the mean IC50 of LSC samples was greater than the predicted plasma concentration and the mean IC50 of blast samples was less than predicted plasma concentration. These data indicate that the observed LSC resistance occurs at clinically relevant concentrations. For daunorubicin, the opposite pattern was observed and for mitoxantrone, mean IC50s for both LSCs and blasts were less than predicted plasma concentration (FIG. 9).

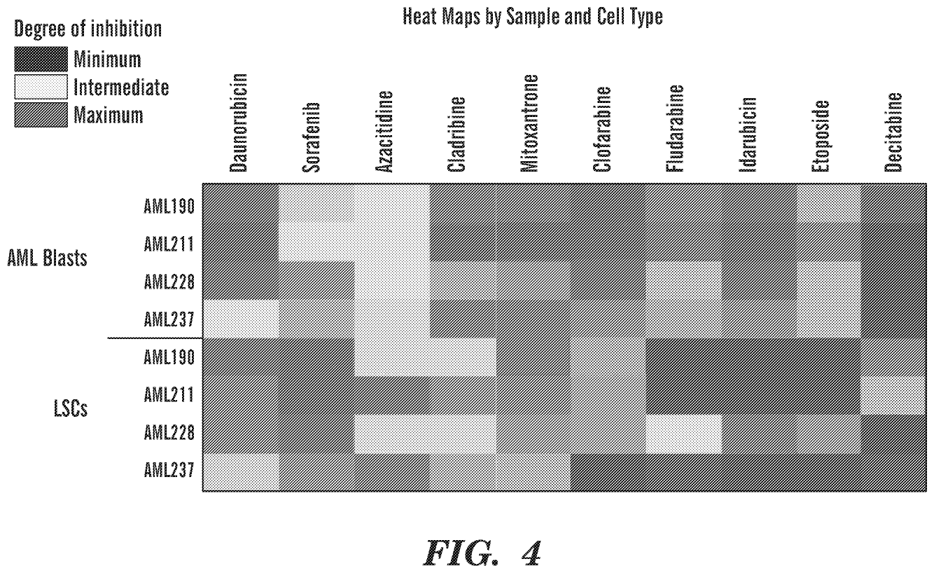

Examination of viability data for individual patient samples, clearly showed relative resistance of LSCs to standard of care AML chemotherapy drugs used in practice today (FIG. 1C). Of note, blast and LSC drug susceptibility patterns were distinct within each patient (FIG. 4).

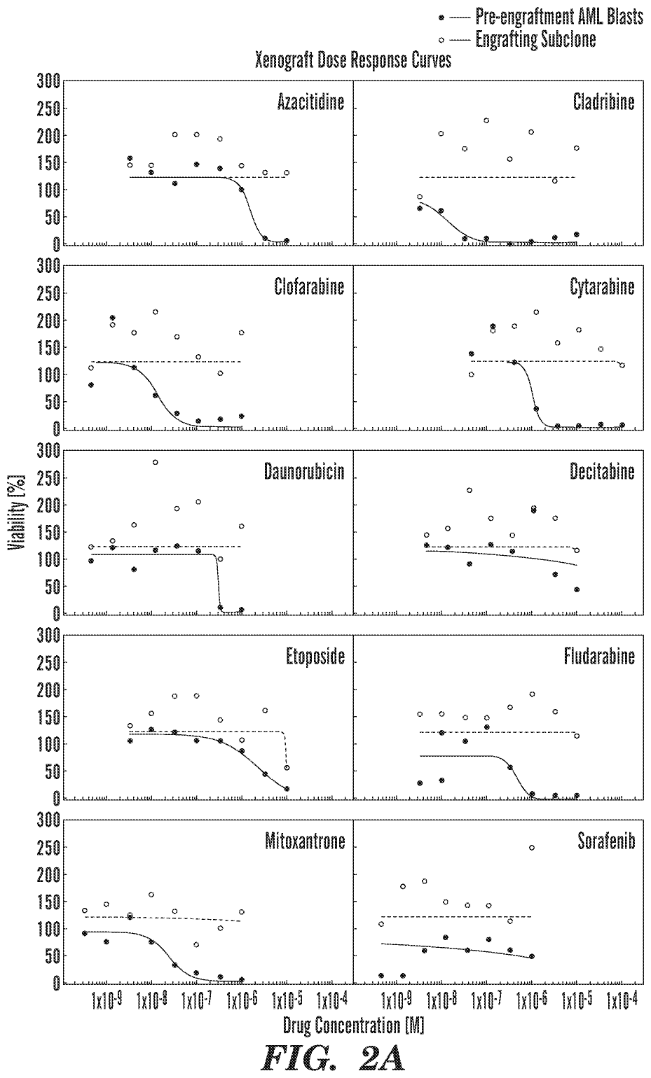

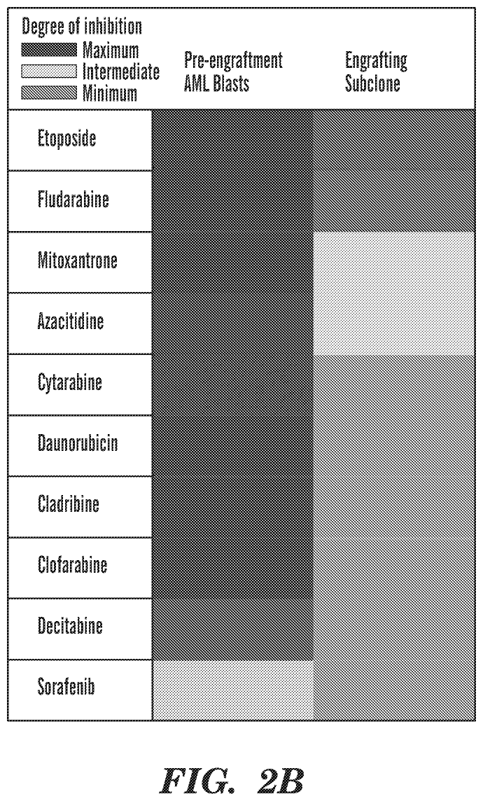

Pre-engraftment blast population vs. engrafting subclone: Comparison of drug susceptibility patterns of the pre-engraftment blast population and the engrafting subclone in our xenograft model showed that the latter was strikingly and divergently resistant to cell cycle-targeted chemotherapy and most drugs (FIG. 2).

Comparison of mutations present in LSCs vs. blasts: In all cases, there was divergence of the mutations present in LSCs vs. blasts from the same patients (Table 5).

| | | | FDA |
|---|---|---|---|
| | | AUC Stem | Approval |
| Compound | Mechanism of Action | Mean | Status |
| Ponatinib | Tyrosine Kinase | 0.00085 | FDA |
| | Inhibitors (TKIs) | | Approved |
| Sorafenib | | 0.0045 | FDA |
| | | | Approved |
| Sunitinib | | 0.00039 | FDA |
| | | | Approved |
| Crenolanib | | 0.0054 | Investigational |
| Romidepsin | Histone Deacetylase | 0.00041 | FDA |
| | Inhibitors (HDACIs) | | Approved |
| Mocetinostat | | 0.0029 | Investigational |
| MG-132 | Proteasome Inhibitors | 0.0039 | Investigational |
| PIK-75 | phosphatidylinositol-3-kinase/ | 0.0041 | Investigational |
| | mammalian target of rapamycin | | |
| | inhibitors (PI3KI/MTORIs) | | |
| YM-155 | Survivin Inhibitor | 0.00089 | Investigational |
| Cabazitaxel | Microtubule | 0.0031 | FDA |
| | Demolymerization | | Approved |
| | Inhibitor | | |
| Dinaciclib | Cyclin-dependent kinase | 0.048 | Investigational |
| | inhbitors (CDKIs) | | |
| SG I-1776 | proto-oncogene proviral | 0.00076 | Investigational |
| | integration site for moloney | | |
| | murine leukemia virus (PIM) | | |
| | Kinase Inhibitor | | |

There were many fewer mutations uniquely present in the leukemia stem cells than in the blast populations. For the mutations shared by the LSCs and the blasts (data not shown), the variant allele frequencies were not significantly different between the two populations. In an effort to identify mutations that contributed to the drug resistance in the LSCs, mutation analysis for the commonly recurring AML mutations was performed. Focusing on mutations with a VAF of 2.5% or higher, there were a few mutations seen uniquely in the LSCs: BUB1 mutations in 2 patients (AML 228 and 211), none in 1 patient, one patient each with KDM2B (AML237) or DEK (AML 190) mutations, one patient with 3 mutations (SRRM2, U2AF2, RBMX) and one patient with 7 mutations. In contrast, there were many more mutations unique to the blasts in every case, ranging from 4 to 67 mutations (Table 3). The BUB1 mutation in AML 211 LSC was c.2212G>T, considered by SIFT deleterious, by Polyphen, possibly damaging. The BUB1 mutation in AML 228 LSC was c.2296G>T, resulted in a premature stop codon. U2AF2 is a splicing factor that is mutated in myelodysplastic syndrome.

### Divergent Drug Susceptibility Patterns: A Mechanism for Relapse and Resistance:

Previous work has suggested relative resistance of the LSC fraction to cell cycle targeting anti-cancer agents; however, these findings have been limited thus far to only a few drugs (e.g. cytarabine, daunorubicin). This study extends these findings by examining drug susceptibility to a panel of FDA approved and investigational anti-cancer drugs. Aggregate viability data identified 40 drugs as blast cell specific (pairwise p-value<0.1), (i.e. blast cells were more susceptible than stem cells), and 32 drugs as leukemia stem cell specific (pairwise p-value<0.1). This indicates that blasts and LSCs have different sensitivities to numerous anti-cancer agents. Among the 11 drugs commonly used to treat AML, 5 (cladribine, idarubicin, etoposide, fludarabine, cytarabine) were blast cell-specific and 2 (clofarabine and mitoxantrone) trended towards blast cell-specific while only 2 (daunorubicin, decitabine) were non-specific and 2 were LSC-specific (sorafenib and azacitidine). Further, the typical chemotherapy agents used to treat AML were significantly more effective against blasts than LSCs. These results underscore the importance of performing functional drug assays to identify drugs with the potential to eliminate the LSCs (FIG. 1).

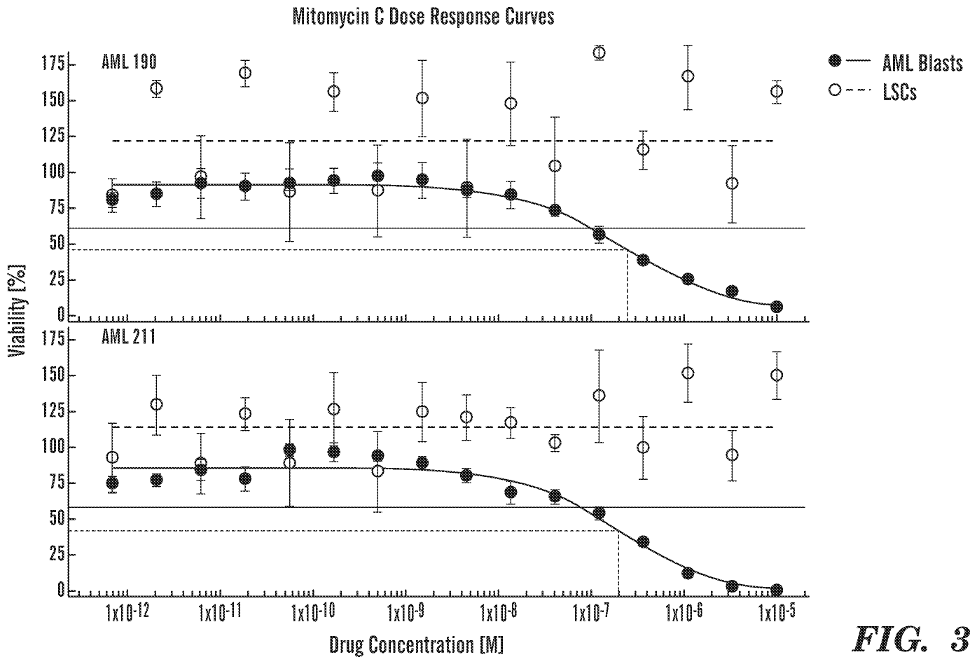

One striking finding was the appearance of the mitomycin C standard curve performed in quadruplicate for all AML blast samples could not be produced by curve fitting for LSCs, where the appearance was that of a scattergram. It was found that this drug appeared to be blast cell specific (pairwise p-value<0.05) (FIG. 1) and LSCs appeared resistant to mitomycin C (FIG. 3). LSC resistance to mitomycin C may reflect a failure of LSCs to take up the drug, efficient protection from DNA damage, enhanced DNA damage repair, or another mechanism.

In addition to FACS isolated LSC populations, the drug resistance patterns of a subclone isolated from a NOD/SCID IL2R γc−/− mouse engrafted with AML blasts from a patient with Flt3 positive AML were investigated. At week 3, one of 29 mice had robust (91%) engraftment of human AML cells while other mice showed much lower levels of engraftment (FIG. 5). Comparison of the pre-engraftment blast population and this engrafting subclone drug sensitivity data showed that the latter was divergently resistant to cell cycle-targeted chemotherapy and the majority of drugs (FIG. 2); this finding was comparable to that observed in the LSCs in the patient samples (FIG. 1).

In contrast to the classical description of an LSC as “quiescent”, the rapid engrafting leukemia cells exhibit a unique phenotype, combining broad resistance with an apparently rapid net growth rate (proliferation minus death rate) as evidenced by the rapidity with which it grew to 91% of the host CD45+ population. It is widely assumed that there is always a “phenotypic cost” of drug resistance, such that drug resistant subclones will have lower net proliferative fitness; however, exceptions are known. Theoretical studies of other deleterious mutations suggest that even if the majority reduce fitness, a minority may increase fitness and lead to rapid exponential growth to predominate in the leukemic cell population. An alternative mechanism for drug resistance is epigenetic alteration that results in change of gene expression.

There can be subclonal “skewing” or “restriction” following engraftment of NOD/SCID IL2R γc−/− and NOD/SCID IL2R γc−/−-SGM3 mice with de novo human AML samples. Engrafting subclones were often rare in starting samples, and displayed variable retention of genetic mutations observed in the pre-engraftment blast sample. As gene mutation patterns are increasingly used to guide treatment strategies, xenotransplantation may provide a way to identify recurrent mutations that govern LSC proliferative behavior and/or drug responsiveness.

Molecular Analysis of LSC Compared to Blast Populations from the Same Patients

Regarding which mutations were seen unique to the LSCs, one patient had a mutation of lysine-specific demethylase 2B (KDM2B). KDM2B controls stem cell self-renewal, senescence, and tumorigenesis. It is a component of the polycomb repressive complex 1 and thereby can target c-Fos for polyubiquitylation and degradation and thus suppress cell proliferation. A second patient had a mutation in the LSCs of BUB1, a protein involved in mitotic checkpoint control. A third patient had a mutation in the LSCs of DEK, a protein involved in proliferation and maintenance of stem cell phenotype that is involved in the t(6;9) translocation that results in a fusion with NUP214 and confers poor prognosis. In each case, there were many more mutations present uniquely in the respective blast populations, likely reflecting clonal evolution. The relationship between the presence of mutations such as these, unique in blast populations, and drug resistance is not certain, but each of these genes is involved in control of proliferation and/or transcription.

### Emerging LSC Effective Anti-Cancer Agents

Aggregate viability data derived from patient samples was used to identify 12 drugs that effectively killed LSCs, and an additional 141 drugs to which LSCs were resistant. Effective drugs included TKIs, HDACIs, 1 CDK inhibitor, 1 proteasome inhibitor and 1 microtubule assembly inhibitor amongst others. Several of the drugs that efficiently killed LSCs have been studied clinically in AML, while others have theoretical or established efficacy vs. LSCs by drug class. Only one commonly used drug in AML, sorafenib, a multikinase inhibitor used in FLT3+ disease that may improve survival in younger patients, was effective against LSCs. HDAC inhibition selectively depletes LSCs in vitro, and the HDAC inhibitor valproate selectively decreases the LSC fraction in AML patients. However, while the LSC effective HDACs in this assay (romidepsin and mocetinostat) have been studied clinically in AML with variable outcomes, their efficacy against LSCs has not been addressed.

Proteasome inhibitors have theoretical potency vs. LSCs through indirect inhibition on NFkB, which is constitutively expressed in LSCs; this anti-LSC effect was shown in vitro with the proteasome inhibitor ixazomib and was confirmed in this study. The proteasome inhibitor bortezomib, ineffective vs. LSCs in this study, has been combined with standard chemotherapy. A later generation proteasome inhibitor carfilzomib, was not effective against LSCs in this study, reduced LSCs in vitro in a prior study but has had minimal anti-leukemic effects in patients.

One investigational PI3k/mTOR inhibitor (PIK-75) was effective against LSCs. This correlates with prior studies using xenograft models, which have associated this pathway with LSC survival. To date, PI3k/mTOR inhibitors have had limited clinical efficacy in AML, but several in class drugs (not tested in this assay) augmented the effect of parthenolide (an NFkB inhibitor) vs. LSCs in vitro. YM-155, a survivin inhibitor effective vs. LSCs in this study is an interesting potential therapy. In AML, survivin is highly expressed in LSCs and correlates with survival. While YM-155 has been tested clinically for other cancers, its effect in AML is unknown. Cabazitaxel, a microtubular depolymerization inhibitor that is effective vs. LSCs here, is used in castration-resistant prostate cancer. It has not yet been tested clinically in adult patients with AML and its effect vs. LSCs had not yet been tested. However, it was effective vs. pediatric AML blasts in vitro.

The results described herein demonstrate unique and distinct patient-specific blast and LSC drug response patterns. This phenotypic diversity supports an approach to treatment that incorporates both blast and LSC specific drug susceptibility data. Simulations of highly adaptive personalized therapy suggest that resistant subclones should be prioritized as therapeutic targets regardless of frequency to avoid the development of multiply-resistant clones with high proliferative potential. One such minority LSC subclone with broad drug resistance and rapid net growth was identified in this study. Future work based on patient-specific LSC and blast drug susceptibility patterns and their evolution during treatment has the potential to identify more effective therapy.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are systems and methods incorporating a a display system for identifying antibody generation, compositions arising from the described systems and methods, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### REFERENCES

The following references are each incorporated herein by reference in their entirety.
Hope K J, Jin L, Dick J E. Acute myeloid leukemia originates from a hierarchy of leukemic stem cell classes that differ in self-renewal capacity. Nature immunology 2004; 5: 738-743.
Ishikawa F, Yoshida S, Saito Y, Hijikata A, Kitamura H, Tanaka S et al. Chemotherapy-resistant human AML stem cells home to and engraft within the bone-marrow endosteal region. Nature Biotechnology 2007; 25: 1315-1321.
Al-Asadi M G, Brindle G, Castellanos M, May S T, Mills K I, Russell N H et al. A molecular signature of dormancy in CD34+CD38-acute myeloid leukaemia cells. Oncotarget 2017; 8: 111405-111418.
Costello R T, Mallet F, Gaugler B, Sainty D, Arnoulet C, Gastaut J A et al. Human acute myeloid leukemia CD34+/CD38− progenitor cells have decreased sensitivity to chemotherapy and Fas-induced apoptosis, reduced immunogenicity, and impaired dendritic cell transformation capacities. Cancer Research 2000; 60: 4403-4411.
Sarry J E, Murphy K, Perry R, Sanchez P V, Secreto A, Keefer C et al. Human acute myelogenous leukemia stem cells are rare and heterogeneous when assayed in NOD/SCID/IL2Rγc-deficient mice. J Clin Invest. 2011; 121, 384-395.
Gerber J M, Smith B D, Ngwang B, Zhang H, Vala M S, Morsberger L et al. A clinically relevant population of leukemic CD34(+) CD38(−) cells in acute myeloid leukemia. Blood 2012; 119:3571-3577.
van Rhenen A, Feller N, Kelder A, Westra A H, Rombouts E, Zweegman S et al. High stem cell frequency in acute myeloid leukemia at diagnosis predicts high minimal residual disease and poor survival. Clin. Cancer Res. 2005; 11: 6520-6527.
Vergez F, Green A S, Tamburini J, Sarry J E, Gaillard B, Cornillet-Lefebvre P et al. High levels of CD34+ CD38low/− CD123+ blasts are predictive of an adverse outcome in acute myeloid leukemia: a Groupe Ouest-Est des Leucémies Aigues et Maladies du Sang (GOELAMS) study. Haematologica 2011, 96(12), 1792-1798.
Roshal M, Chien S, Othus M, Wood B L, Fang M, Appelbaum F R et al. The proportion of CD34(+)CD38(low or neg) myeloblasts, but not side population frequency, predicts initial response to induction therapy in patients with newly diagnosed acute myeloid leukemia. Leukemia 2013; 27: 728-731.
Ng S W, Mitchell A, Kennedy J A, Chen W C, McLeod J, Ibrahimova N et al. A 17-gene stemness score for rapid determination of risk in acute leukaemia. Nature 2016, 540: 433-437.
Gentles A J, Plevritis S K, Majeti R, Alizadeh A A. Association of a leukemic stem cell gene expression signature with clinical outcomes in acute myeloid leukemia. JAMA 2010; 304: 2706-2715.
McDermott S P, Eppert K, Notta F, Isaac M, Datti A, Al-Awar R et al. A small molecule screening strategy with validation on human leukemia stem cells uncovers the therapeutic efficacy of kinetin riboside. Blood 2012; 119:1200-1207.
Nieborowska-Skorska M, Sullivan K, Dasgupta Y, Podszywalow-Bartnicka P, Hoser G, Maifrede S et al. Gene expression and mutation-guided synthetic lethality eradicates proliferating and quiescent leukemia cells. J Clin Invest. 2017; 127:2392-2406.
Taussig, D C, Miraki-Moud F, Anjos-Afonso F, Pearce D J, Allen K, Ridler C et al. Anti-CD38 antibody-mediated clearance of human repopulating cells masks the heterogeneity of leukemia-initiating cells. Blood 2008; 112: 568-575.
Jordan C T, Upchurch D, Szilvassy S J, Guzman M L, Howard D S, Pettigrew A L et al. The interleukin-3 receptor alpha chain is a unique marker for human acute myelogenous leukemia stem cells. Leukemia 2000; 14: 1777-1784.
Jin L, Lee E M, Ramshaw H S, Busfield S J, Peoppl A G, Wilkinson L et al. Monoclonal antibody-mediated targeting of CD123, IL-3 receptor alpha chain, eliminates human acute myeloid leukemic stem cells. Cell Stem Cell 2009; 5:31-42.
McKenzie J L, Gan O I, Doedens M, Dick J E. Reversible cell surface expression of CD38 on CD34-positive human hematopoietic repopulating cells. Exp Hematol. 2007; 35:1429-36.
Beckman R A, Loeb L A. Efficiency of carcinogenesis with and without a mutator mutation. Proceedings of the National Academy of Sciences 2006; 103: 14140-14145.
Pemovska T, Kontro M, Yadav B, Edgren H, Eldfors S, Szwajda A et al. Individualized systems medicine strategy to tailor treatments for patients with chemorefractory acute myeloid leukemia. Cancer Discovery 2013; 3: 1416-1429.
Beckman R A, Schemmann G S, Yeang C H. Impact of genetic dynamics and single-cell heterogeneity on development of nonstandard personalized medicine strategies for cancer. Proceedings of the National Academy of Sciences 2012; 109: 14586-14591.
Klco J M, Spencer D H, Miller C A, Griffith M, Lamprecht T L, O'Laughlin M et al. Functional heterogeneity of genetically defined subclones in acute myeloid leukemia. Cancer Cell 2014; 25: 379-392.
Lichtman M A, A historical perspective on the development of the cytarabine (7 days) and daunorubicin (3 days) treatment regimen for acute myelogenous leukemia: 2013 the 40th anniversary of 7+3. Blood Cells, Mol Dis 2013; 50: 119-130.
Becker P S, Chien S, Martins T J, Herstein A, Hammer C, Sekizaki T et al. A precision medicine approach Incorporating both molecular and in vitro functional data to treat patients with relapsed/refractory acute myeloid leukemia. Blood 2016; 128: 4043 (abstract).
Swords R T, Azzam D, Al-Ali H, Lohse I, Volmar C H, Watts J M, et al. Ex-vivo sensitivity profiling to guide clinical decision making in acute myeloid leukemia: A pilot study. Leuk Res. 2018; 64:34-41.
Gerby B, Veiga D F, Krosl J, Nourreddine S, Ouellette J, Haman A et al. High-throughput screening in niche-based assay identifies compounds to target preleukemic stem cells. J. Clin. Invest. 2016; 126: 4569-4584.
Kihara R, Nagata Y, Kiyoi H, Kato T, Yamamoto E, Suzuki K et al. Comprehensive analysis of genetic alterations and their prognostic impacts in adult acute myeloid leukemia patients. Leukemia 2014; 28: 1586-1595.
Rao R C, Dou Y. Hijacked in cancer: the MLL/KMT2 family of methyltransferases. Nature Rev Cancer 2015; 15: 334-346.
Zhou M H, Yang Q M. NUP214 fusion genes in acute leukemia. Oncology Lett. 2014; 8: 959-962.
Schmitt, M W, Fox E J, Prindle M J, Reid-Bayliss K S, True L D, Radich J P et al. Sequencing small genomic targets with high efficiency and extreme accuracy. Nature Meth 2015; 12: 423-425.
Lee S I, Celik S, Logsdon B A, Lundberg S M, Martins T J, Oehler V G et al. A machine learning approach to integrate big data for precision medicine in acute myeloid leukemia. Nat. Commun. 2018; 9: 42.
Gatenby R A, Silva A S, Gillies R J, Frieden B R. Adaptive therapy. Cancer Res 2009; 69: 4894-4903.
Shah N P, Skaggs B J, Branford S, Hughes T P, Nicoll J M, Paquette R L et al. Sequential ABL kinase inhibitor therapy selects for compound drug-resistant BCR-ABL mutations with altered oncogenic potency. J. Clin. Invest. 2007; 117: 2562-2369.
Beckman R A, Loeb L A. Negative clonal selection in tumor evolution. Genetics 2005; 171: 2123-2131.
Fong C Y, Gilan O, Lam E Y, Rubin A F, Ftouni S, Tyler D, et al. BET inhibitor resistance emerges from leukaemia stem cells. Nature 2015; 525: 538-542.
Ravandi F, Alattar M L, Grunwald M R, Rudek M A, Rajkhowa T, Richie M A et al. Phase 2 study of azacytidine plus sorafenib in patients with acute myeloid leukemia and FLT-3 internal tandem duplication mutation. Blood 2013; 121: 4655-4662.
Qi, J., Singh, S., Hua, W. K., Cai, Q., Chao, S. W., Li, L. et al. (2015). HDAC8 inhibition specifically targets Inv (16) acute myeloid leukemic stem cells by restoring p53 acetylation. Cell Stem Cell 2015; 17: 597-610.
Craddock C, Quek L, Goardon N, Freeman S, Siddique S, Raghavan M et al. (2013). Azacitidine fails to eradicate leukemic stem/progenitor cell populations in patients with acute myeloid leukemia and myelodysplasia. Leukemia 2013; 27: 1028-1036.
Luger S M, O'Connell C L, Klimek V, Cooper M A, Besa E C, Rossetti J M et al. A phase 2 study of mocetinostat, an oral isotype-selective histone deacetylase (HDAC) inhibitor, in combination with 5-azacitidine in patients with myelodysplastic syndrome (MDS). J. Clin. Oncol. 2013; 31 (suppl): 7116 (abstract).
Klimek V M, Fircanis S, Maslak P, Guernah I, Baum M, Wu N et al. Tolerability, pharmacodynamics, and pharmacokinetics studies of depsipeptide (romidepsin) in patients with acute myelogenous leukemia or advanced myelodysplastic syndromes. Clinical Cancer Research 2008; 14: 826-832.
Kirschbaum M H, Foon K A, Frankel P, Ruel C, Pulone B, Tuscano J M et al. A phase 2 study of belinostat (PXD101) in patients with relapsed or refractory acute myeloid leukemia or patients over the age of 60 with newly diagnosed acute myeloid leukemia: a California Cancer Consortium Study. Leuk. Lymphoma 2014; 55: 2301-2304.
Guzman M L, Swiderski C F, Howard D S, Grimes B A, Rossi R M, Szilvassy S J et al. Preferential induction of apoptosis for primary human leukemic stem cells. Proc. Natl. Acad. Sci. USA 2002; 99: 16220-16225.
Attar E C, DeAngelo D J, Supko J G, D'Amato F, Zahrieh D, Sirulnik A et al. Phase I and pharmacokinetic study of bortezomib in combination with idarubicin and cytarabine in patients with acute myelogenous leukemia. Clin. Cancer Res. 2008; 14: 1446-1454.
Helm L H, Bosman M C, Schuringa J J, Vellenga E. Effective targeting of primitive AML CD34+ cells by the second-generation proteasome inhibitor carfilzomib. Br. J. Haematol. 2015; 171: 652-655.
Wartman L D, Fiala M A, Fletcher T, Hawkins E R, Cashen A, DiPersio J F et al. A phase I study of carfilzomib for relapsed or refractory acute myeloid and acute lymphoblastic leukemia. Leuk. Lymphoma 2016; 57: 728-730.
Martelli A M, Evangelisti C, Chappell W, Abrams S L, Basecke J, Stivala, F et al. Targeting the translational apparatus to improve leukemia therapy: roles of the PI3K/PTEN/Akt/mTOR pathway. Leukemia 2011; 25: 1064-1079.
Yee K W, Zeng Z, Konopleva M, Verstovsek S, Ravandi F, Ferrajoli A et al. Phase I/II study of the mammalian target of rapamycin inhibitor everolimus (RAD001) in patients with relapsed or refractory hematologic malignancies. Clin. Cancer Res. 2006; 12: 5165-5173.
Hassane D C, Sen S, Minhajuddin M, Rossi R M, Corbett C A, Balys M et al. Chemical genomic screening reveals synergism between parthenolide and inhibitors of the PI-3 kinase and mTOR pathways. Blood 2010; 116: 5983-5990.
Carter B Z, Qiu Y, Huang X, Diao L, Zhang N, Coombes K R et al. Survivin is highly expressed in CD34+ 38− leukemic stem/progenitor cells and predicts poor clinical outcomes in AML. Blood 2012; 120: 173-180.
Smith A M, Little E B, Zivanovic A, Hong P, Liu A K, Burow R et al. Targeting survivin with YM155 (Sepantronium Bromide): A novel therapeutic strategy for paediatric acute myeloid leukaemia. Leukemia Res. 2015; 39: 435-444.
Drenberg C D, Shelat A, Orwick S J, Inaba H, Ribeiro R C, Rubnitz J E et al. Integrated high-throughput screen to identify treatment leads for pediatric AML. Cancer Res. 2016; 76 (Suppl): 4786 (Abstract).

1. (canceled)

2. A high throughput functional cell assay comprising the steps of:
(a) contacting aliquots of a biological sample from an individual having cancer with individual members of a panel of anti-cancer agents, the sample comprising a population of non-stem cell cancer cells and a population of cancer stem cells (CSCs); and
(b) quantifying, respectively, cell viability of the population of CSCs and the population of non-stem cell cancer cells (NSCCCs).

3. The assay of claim 2, wherein cell viability is assessed using a tetrazolium reduction assay, a resazurin reduction assay, a protease viability marker assay, a live cell protease assay, an ATP assay, a luciferase-based real-time assay, flow cytometry, or high content imaging.

4. The assay of claim 2, further comprising a step, performed prior to steps (a) and (b), of seeding the aliquots of the biological sample in a plurality of wells.

5. (canceled)

6. The assay of claim 2, wherein the panel of anti-cancer agents comprises at least 2 anti-cancer agents and no more than 1000 anti-cancer agents.

7. The assay of claim 2, wherein each individual member of the panel is tested with at least five different concentrations of the anti-cancer agent for each population.

8. The assay of claim 7, wherein a dose-response curve is generated for each individual member and each population using the data from the at least two different concentrations of the anti-cancer agent.

9. The assay of claim 8, wherein Area Under the Curve (AUC) is calculated for each individual member in the panel of anti-cancer agents and for each population from the respective dose-response curve for that anti-cancer agent.

10. (canceled)

11. The assay of claim 2, further comprising a step of ranking the individual members of the panel of anti-cancer agents based on their effect on cell viability for CSCs and/or NSCCCs.

12. The assay of claim 2, further comprising a step of comparing the cell viability for each anti-cancer agent on CSCs and/or NSCCCs to a reference.

13. The assay of claim 2, wherein quantifying step (b) comprises detecting signal from one or more markers permitting quantitative distinction between populations of CSCs and NSCCCs.

14. The assay of claim 2, further comprising a step of contacting the population of NSCCCs and the population of cancer stem cells (CSCs) with detectable probes that specifically bind and provide signal for the one or more markers.

15. (canceled)

16. (canceled)

17. A method for selecting a personalized treatment for a subject having cancer, the method comprising:
(a) performing a high throughput functional cell assay of claim 2 on a biological sample from a subject having cancer; and
(b) on the basis of cell viability determined for CSCs and NSCCCs in (a), selecting a combination of at least two anti-cancer agents from the panel, the combination comprising a drug(s) effective to kill CSCs and a drug(s) effective to kill NSCCCs, thereby selecting a personalized treatment for the subject.

18. The method of claim 17, further comprising administering the combination of anti-cancer agents to the subject, thereby treating the subject's cancer.

19. The method of claim 17, wherein the cancer is refractory to or the subject has relapsed from prior treatment with a given anti-cancer agent.

20. (canceled)

21. A high throughput functional cell assay comprising the steps of:
(a) isolating from a biological sample obtained from a subject having cancer, a population enriched for viable cancer stem cells (CSCs),
(b) isolating from the same or a different biological sample obtained from the subject having cancer, a population enriched for viable non-stem cell cancer cells (NSCCCs);
(c) contacting aliquots of the population enriched for CSCs with individual members of a panel of anti-cancer agents,
(d) contacting aliquots of the population enriched for NSCCCs with individual members of a panel of anti-cancer agents, and
(e) determining cell viability of the cells in each of the populations of step (c) and (d).

22.-52. (canceled)

53. A method for monitoring treatment efficacy in a subject being treated for cancer, the method comprising:
(a) isolating from a biological sample obtained from a subject being treated for cancer with an anti-cancer agent, a population enriched for cancer stem cells (CSCs),
(b) isolating from the same or a different biological sample obtained from the subject being treated for cancer with an anti-cancer agent(s), a population enriched for cancer cells;
(c) contacting aliquots of the population enriched for CSCs with the anti-cancer agent(s),
(d) contacting aliquots of the population enriched for non-stem cell cancer cells with the anti-cancer agents(s), and
(e) determining cell viability of the cells in each of the populations of step (c) and (d),
wherein a reduction in cell viability in the presence of the anti-cancer agent as compared to an untreated or vehicle treated aliquot of the same cell population indicates that the anti-cancer is efficacious in the subject being treated for cancer.

54. The method of claim 53, wherein the method is repeated at least once while the subject is being treated for cancer.

55. The method of claim 53, wherein the method is repeated weekly, monthly, every 6 months, or annually.

56. The method of claim 53, further comprising the steps of:
(a) contacting aliquots of the population enriched for CSCs with individual members of a panel of anti-cancer agents,
(b) contacting aliquots of the population enriched for cancer cells with individual members of a panel of anti-cancer agents,
(c) determining cell viability of the cells in each of the populations of step (a) and (b),
(d) selecting, based on criteria comprising reduced cell viability assessed in step (c), the same or a different anti-cancer agent or combination thereof than the anti-cancer agent used to treat cancer in the subject.

57.-81. (canceled)

82. The method of claim 21, further comprising a step of selecting, based on criteria comprising reduced cell viability, at least one anti-cancer agent, thereby selecting a personalized treatment for the subject having cancer.
